(19) United States
(12) Patent Application Publication
(10) Pub. No.: US 2020/0330400 A1
(43) Pub. Date: Oct. 22, 2020

(54) METHODS FOR THE TREATMENT OF CANCER USING COENZYME Q10 AND FATTY ACID METABOLISM INHIBITORS

(71) Applicant: Berg LLC, Framingham, MA (US)

(72) Inventors: Stephane Gesta, Arlington, MA (US); Anne R. Diers, Wilmington, MA (US); Tulin Dadali-Abel, Jamaica Plain, MA (US)

(21) Appl. No.: 16/085,395

(22) PCT Filed: Mar. 16, 2017
(86) PCT No.: PCT/US2017/022839

§ 371 Date: Sep. 14, 2018

Related U.S. Application Data
(60) Provisional application No. 62/324,156, filed on Apr. 18, 2016.
(60) Provisional application No. 62/309,295, filed on Mar. 16, 2016.

(51) Int. Cl. A61K 31/122; A61K 9/00; A61K 47/24; C12N 15/113; A61K 31/365; A61K 31/445; A61P 35/00; A61K 31/336

(52) U.S. Cl. CPC A61K 31/122; A61K 9/0014; A61K 9/0019; A61K 9/007; A61K 31/336; C12N 15/113; A61K 31/365; A61K 31/445; A61P 35/00; A61K 47/24

(57) ABSTRACT

Presented herein are methods for the treatment of oncological disorders by the co-administration of CoQ10 compositions and at least one fatty acid metabolism inhibitor. In one embodiment, the CoQ10 compositions are lipid-containing compositions. The fatty acid metabolism inhibitor may be an inhibitor of fatty acid synthesis, storage, transport or degradation. The fatty acid metabolism inhibitor may also be a modulator of fatty acid structure, for example a fatty acid desaturase or elongase. The fatty acid inhibitor may inhibit any molecule involved in fatty acid metabolism, such as fatty acid synthase (FASN), carnitine palmitoyltransferase 1 (CPT-1), long-chain 3-ketoacyl-CoA thiolase, or stearoyl-CoA desaturase-1 (SCD-1). In embodiments, the fatty acid metabolism inhibitor may be C75, Etomoxir, trimetazidine or A939572.

### RELATED APPLICATIONS

This application claims priority to U.S. Provisional Patent Application No. 62/309,295 filed on Mar. 16, 2016, and U.S. Provisional Patent Application No. 62/324,156 filed on Apr. 18, 2016, the contents of each of which are incorporated herein in their entirety.

### FIELD OF THE INVENTION

The invention generally relates to methods for the treatment of oncological disorders comprising administration of coenzyme Q10 (CoQ10) and one or more fatty acid metabolism inhibitors.

### BACKGROUND

Cancer is presently one of the leading causes of death in developed nations. A diagnosis of cancer traditionally involves serious health complications. Cancer can cause disfigurement, chronic or acute pain, lesions, organ failure, or even death. Commonly diagnosed cancers include pancreatic cancer, breast cancer, lung cancer, melanoma, lymphoma, carcinoma, sarcoma non-Hodgkin's lymphoma, leukemia, endometrial cancer, colon and rectal cancer, prostate cancer, and bladder cancer. Traditionally, many cancers (e.g., breast cancer, leukemia, lung cancer, or the like) are treated with surgery, chemotherapy, radiation, or combinations thereof. Chemotherapeutic agents used in the treatment of cancer are known to produce several serious and unpleasant side effects in patients. For example, some chemotherapeutic agents cause neuropathy, nephrotoxicity (e.g., hyperlipidemia, proteinuria, hypoproteinemia, combinations thereof, or the like), stomatitis, mucositisemesis, alopecia, anorexia, esophagitis amenorrhoea, decreased immunity, anaemia, high tone hearing loss, cardiotoxicity, fatigue, neuropathy, or combinations thereof. Improved methods for the treatment of cancer, and composition capable of delivering bioactive agents to aid in the treatment of diseases and other conditions remain desirable.

### SUMMARY OF THE INVENTION

In certain aspects, the present inventions relate to combination therapies comprising administration of Coenzyme Q10 and at least one fatty acid metabolism inhibitor for the treatment of oncological disorders. Applicants have surprisingly found that cancer cells, such as breast cancer and pancreatic cancer cells, were more sensitive to Coenzyme Q10 when treated in combination with a fatty acid metabolism inhibitor, such as C75, Etomoxir, and trimetazidine. In addition, pancreatic cancer cells were more sensitive to Coenzyme Q10 when treated in combination with the fatty acid metabolism inhibitor A939572, an inhibitor of Stearoyl-CoA desaturase-1 (SCD-1).

Furthermore, Coenzyme Q10 treatment was associated with a dose- and time-dependent increase in mRNA expression of the fatty acid metabolism enzymes fatty acid synthase (FASN), carnitine palmitoyltransferase 1 (CPT-1), long-chain-fatty-acid—CoA ligase 1 (ACSL1), and Diglyceride acyltransferase (DGAT1) in breast cancer cells. These enzymes are involved in several aspects of fatty acid metabolism, such as fatty acid synthesis, transport, storage and degradation. Accordingly, these results indicate that a broad range of fatty acid metabolism inhibitors may be used to sensitize cancer cells to Coenzyme Q10.

To further evaluate the effect of Coenzyme Q10 on cancer cell lipid composition, structural lipidomic analysis was used to assess the metabolic fate of the lipid components of a phospholipid-containing Coenzyme Q10 formulation. Structural lipidomic analysis demonstrated that these lipid components were readily incorporated into prevalent diacyl-glycerol (DAG) and triacyl-glycerol (TAG) species along with de novo fatty acid species such as palmitate in breast cancer cells. These results demonstrate that lipid components of the phospholipid-containing Coenzyme Q10 formulation are readily accumulated in cancer cells and incorporated into DAG and TAG species, indicating that cancer cells actively respond to the addition of exogenous lipids to increase their storage or degradation. Accordingly, while not wishing to be bound by theory, cancer cells treated with lipid-containing formulations, e.g., CoQ10 formulations containing a lipid (e.g., phospholipid), may be especially sensitive to fatty acid metabolism inhibitors, due to the increased fatty acid metabolism activity resulting from storage and degradation of the lipid formulation components.

Accordingly, in certain aspects, the invention provides a method of treating an oncological disorder in a subject comprising administering Coenzyme Q10 (CoQ10) and at least one fatty acid metabolism inhibitor to the subject, thereby treating the oncological disorder in the subject. In certain embodiments, a response of the oncological disorder to treatment is improved relative to treatment with the at least one fatty acid metabolism inhibitor alone. In certain embodiments, a response of the oncological disorder to treatment is improved relative to treatment with CoQ10 alone. In certain embodiments, the response is improved by at least 5%, at least 10%, at least 15%, at least 20%, at least 30%, at least 40% or at least 50% relative to treatment with the at least one fatty acid metabolism inhibitor alone. In certain embodiments, the response is improved by at least 5%, at least 10%, at least 15%, at least 20%, at least 30%, at least 40% or at least 50% relative to treatment with Coenzyme Q10 alone. In certain embodiments, the response comprises any one or more of reduction in tumor burden, reduction in tumor size, inhibition of tumor growth, achieving stable oncological disorder in a subject with a progressive oncological disorder prior to treatment, increased time to progression of the oncological disorder, and increased time of survival.

In certain aspects the invention provides a method of treating an oncological disorder in a subject comprising: administering Coenzyme Q10 (CoQ10) to the subject; and administering at least one fatty acid metabolism inhibitor to the subject at a dosage that is lower than standard dosages of the fatty acid metabolism inhibitor used to treat the oncological disorder, thereby treating the oncological disorder.

In certain aspects the invention provides a method of treating an oncological disorder in a subject comprising: administering at least one fatty acid metabolism inhibitor to the subject; and administering Coenzyme Q10 (CoQ10) to the subject at a dosage that is lower than standard dosages of the CoQ10 used to treat the oncological disorder, thereby treating the oncological disorder. In certain embodiments, the CoQ10 is administered topically. In certain embodiments, the CoQ10 is administered by inhalation. In certain embodiments, the CoQ10 is administered by injection or infusion. In certain embodiments, the CoQ10 is administered by intravenous administration. In certain embodiments, the CoQ10 is administered by continuous intravenous infusion. In certain embodiments, the CoQ10 is formulated in a lipid composition. In certain embodiments, the lipid composition comprises: an aqueous solution, CoQ10, and at least one of a dispersion stabilizing agent and an opsonization reducer; wherein the CoQ10 is dispersed into a colloidal nano-dispersion of particles having a mean particle size of less than 200 nm. In certain embodiments, the dispersion stabilizing agent is selected from the group consisting of pegylated castor oil, Cremophor EL, Cremophor RH 40, Pegylated vitamin E, Vitamin E TPGS, and Dimyristoylphosphatidyl choline (DMPC). In certain embodiments, the dispersion stabilizing agent is DMPC.

In certain embodiments of the aforementioned methods, the at least one fatty acid metabolism inhibitor comprises a nucleic acid, a polypeptide, or a small molecule. In certain embodiments, the fatty acid metabolism inhibitor is a nucleic acid inhibitor. In certain embodiments, the nucleic acid inhibitor is an antisense nucleic acid molecule or a double stranded nucleic acid molecule. In certain embodiments, the double stranded nucleic acid molecule is a double stranded RNA selected from the group consisting of an siRNA, a shRNA, and a dicer substrate siRNA (DsiRNA). In certain embodiments, the fatty acid metabolism inhibitor is a polypeptide. In certain embodiments, the polypeptide is an antibody or an antigen-binding fragment thereof. In certain embodiments, the at least one fatty acid metabolism inhibitor comprises a small molecule. In certain embodiments, the small molecule is a carboxylic acid.

In certain embodiments of the aforementioned methods, the at least one fatty acid metabolism inhibitor is an inhibitor of a process in fatty acid metabolism selected from the group consisting of fatty acid synthesis, fatty acid transport and storage, fatty acid degradation, and a modulator of fatty acid structure. In certain embodiments, the at least one fatty acid metabolism inhibitor is an inhibitor of a molecule selected from the group consisting of fatty acid synthase (FASN), carnitine palmitoyltransferase 1 (CPT-1), long-chain 3-ketoacyl-CoA thiolase, and stearoyl-CoA desaturase-1 (SCD-1). In certain embodiments, the at least one fatty acid metabolism inhibitor is an inhibitor of FASN. In certain embodiments, the inhibitor of FASN is C75. In certain embodiments, the at least one fatty acid metabolism inhibitor is an inhibitor of CPT-1. In certain embodiments, the inhibitor of CPT-1 is etomoxir. In certain embodiments, the at least one fatty acid metabolism inhibitor is an inhibitor of long-chain 3-ketoacyl-CoA thiolase. In certain embodiments, the inhibitor of long-chain 3-ketoacyl-CoA thiolase is trimetazidine. In certain embodiments, the at least one fatty acid metabolism inhibitor is an inhibitor of SCD-1. In certain embodiments, the inhibitor of SCD-1 is A939572.

In certain embodiments of the aforementioned methods, the at least one fatty acid metabolism inhibitor is selected from the group consisting of etomoxir, C75, trimetazidine, A939572, cerulenin, 5-(tetradecyloxy)-2-furoic acid, oxfenicine, methyl palmoxirate, metoprolol, amiodarone, perhexiline, aminocarnitine, hydrazonopropionic acid, 4-bromocrotonic acid, ranolazine, hypoglycin, dichloroacetate, methylene cyclopropyl acetic acid, beta-hydroxy butyrate, and a non-hydrolyzable analog of carnitine, and the compounds listed in Tables 2A, 2B, 2C, 2D, 3 or 4. In certain embodiments, the at least one fatty acid metabolism inhibitor is an oxirane carboxylic acid compound having the following formula:

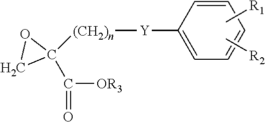

wherein: R1 represents a hydrogen atom, a halogen atom, a 1-4C alkyl group, a 1-4C alkoxy group, a nitro group or a trifluoromethyl group; R2 has one of the meanings of R1; R3 represents a hydrogen atom or a 1-4C alkyl group; Y represents the grouping —O—(CH2)m-; m is 0 or a whole number from 1 to 4; and n is a whole number from 2 to 8 wherein the sum of m and n is a whole number from 2 to 8. In certain embodiments, R1 is a halogen atom, R2 is a hydrogen atom, m is 0, and n is 6, and R3 is an ethyl group.

In certain embodiments, the fatty acid metabolism inhibitor is selected from the group consisting of 2-(6-(4-chlorophenoxy)-hexyl)-oxirane-2-carboxylic acid ethyl ester (etomoxir), 2-(4-(3-chlorophenoxy)-butyl)-oxirane-2-carboxylic acid ethyl ester, 2-(4-(3-trifluoromethylphenoxy)-butyl)-oxirane-2-carboxylic acid ethyl ester, 2-(5(4-chlorophenoxy)-pentyl)-oxirane-2-carboxylic acid ethyl ester, 2-(6-(3,4-dichlorophenoxy)-hexyl)-oxirane-2-carboxylic acid ethyl ester, 2-(6-(4-fluorophenoxy)-hexyl)-oxirane-2-carboxylic acid ethyl ester, and 2-(6-phenoxyhexyl)-oxirane-2-carboxylic acid ethyl ester, the corresponding oxirane carboxylic acids, and their pharmacologically acceptable salts.

In certain embodiments, the at least one fatty acid metabolism inhibitor has the structure:

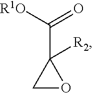

wherein one or both of R1 and R2 are independently an alkyl. In certain embodiments, the alkyl is a straight-chain alkyl. In certain embodiments, R2 has the structure:

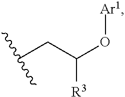

where R3 comprises an organic moiety and Ar1 comprises an aromatic moiety. In certain embodiments, Ar1 is a benzene ring or a derivative thereof having the structure:

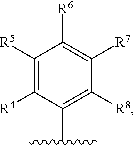

wherein each of R4, R5, R6, R7, and R8 is hydrogen, a halogen, an alkyl, or an alkoxy.

In certain embodiments, the at least one fatty acid metabolism inhibitor has the structure:

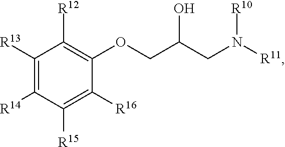

wherein each of R10, R11, R12, R13, R14 R15 and R16 independently comprises hydrogen, a halogen, or an organic moiety.

In certain embodiments, the fatty acid metabolism inhibitor has the structure:

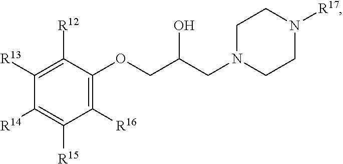

wherein R17 comprises an alkyl, an alkoxy, an aromatic moiety or an amide.

In certain embodiments, the aforementioned methods further comprise administering at least one additional chemotherapeutic agent to the subject. In certain embodiments, the oncological disorder is selected from the group consisting of a carcinoma, sarcoma, lymphoma, melanoma, and leukemia. In certain embodiments, the oncological disorder is selected from the group consisting of pancreatic cancer, breast cancer, ovarian cancer, renal cell carcinoma, liver cancer, skin cancer, lung cancer, colon cancer, prostate cancer, thyroid cancer, bladder cancer, rectal cancer, endometrial cancer, kidney cancer, bone cancer, brain cancer, cervical cancer, stomach cancer, mouth and oral cancers, neuroblastoma, testicular cancer, uterine cancer, and vulvar cancer. In certain embodiments, the oncological disorder is selected from the group consisting of breast cancer, ovarian cancer, pancreatic cancer and renal cell carcinoma. In certain embodiments, the subject is a human.

In certain aspects the invention provides a method of treating an oncological disorder in a subject comprising administering Coenzyme Q10 (CoQ10) and at least one fatty acid metabolism inhibitor to the subject, thereby treating the oncological disorder in the subject. In certain embodiments of the aforementioned method, a response of the oncological disorder to treatment is improved relative to treatment with the at least one fatty acid metabolism inhibitor alone. In certain embodiments, a response of the oncological disorder to treatment is improved relative to treatment with CoQ10 alone. In certain embodiments, the response is improved by at least 5%, at least 10%, at least 15%, at least 20%, at least 30%, at least 40% or at least 50% relative to treatment with the at least one fatty acid metabolism inhibitor alone. In certain embodiments, the response is improved by at least 5%, at least 10%, at least 15%, at least 20%, at least 30%, at least 40% or at least 50% relative to treatment with Coenzyme Q10 alone. In certain embodiments, the response comprises any one or more of reduction in tumor burden, reduction in tumor size, inhibition of tumor growth, achieving stable oncological disorder in a subject with a progressive oncological disorder prior to treatment, increased time to progression of the oncological disorder, and increased time of survival.

In certain embodiments of the aforementioned methods, the CoQ10 is administered topically. In certain embodiments, the CoQ10 is administered by inhalation. In certain embodiments, the CoQ10 is administered by injection or infusion. In certain embodiments, the CoQ10 is administered by intravenous administration. In certain embodiments, the CoQ10 is administered by continuous intravenous infusion. In certain embodiments, the methods further comprise administering at least one additional chemotherapeutic agent to the subject.

In certain aspects, the invention relates to a method of treating an oncological disorder in a subject comprising administering Coenzyme Q10 (CoQ10) to the subject; and administering at least one fatty acid metabolism inhibitor to the subject at a dosage that is lower than standard dosages of the fatty acid metabolism inhibitor used to treat the oncological disorder, thereby treating the oncological disorder. In certain embodiments, the CoQ10 is administered topically. In certain embodiments, the CoQ10 is administered by inhalation. In certain embodiments, the CoQ10 is administered by injection or infusion. In certain embodiments, the CoQ10 is administered by intravenous administration. In certain embodiments, the CoQ10 is administered by continuous intravenous infusion. In certain embodiments, the methods further comprise administering at least one additional chemotherapeutic agent to the subject.

In certain embodiments of the aforementioned methods, the at least one fatty acid metabolism inhibitor is selected from the group consisting of C75, Etomoxir, trimetazidine, inhibitors of fatty acid synthase (FASN), carnitine palmitoyltransferase 1 (CPT-1) and a β-oxidation inhibitor. In certain embodiments, the at least one fatty acid metabolism inhibitor is selected from the group consisting of C75, Etomoxir and trimetazidine. In certain embodiments, the oncological disorder is selected from the group consisting of a carcinoma, sarcoma, lymphoma, melanoma, and leukemia. In certain embodiments, the oncological disorder is selected from the group consisting of pancreatic cancer, breast cancer, liver cancer, skin cancer, lung cancer, colon cancer, prostate cancer, thyroid cancer, bladder cancer, rectal cancer, endometrial cancer, kidney cancer, bone cancer, brain cancer, cervical cancer, stomach cancer, mouth and oral cancers, neuroblastoma, testicular cancer, uterine cancer, and vulvar cancer.

In certain embodiments, the oncological disorder is breast cancer.

In certain embodiments of the aforementioned methods, the subject is human.

### BRIEF DESCRIPTION OF THE DRAWINGS

FIG. 1A shows the effects of various concentrations of Coenzyme Q10 (CoQ10) alone or in combination with the fatty acid metabolism inhibitor C75 (5 μM) on cell viability in MDA-MB231 (upper panel) or SKBr-3 (lower panel) breast cancer cells. Figure IB shows the effects of various concentrations of Coenzyme Q10 (CoQ10) alone or in combination with the fatty acid metabolism inhibitor C75 (12 μM) on cell viability in SKBr-3 breast cancer cells. “a” indicates a statistically significant difference (p<0.05) between cells treated with fatty acid metabolism inhibitor alone and cells treated with combinations of fatty acid metabolism inhibitor and Coenzyme Q10. The asterisks indicate a statistically significant difference between Coenzyme Q10 alone and Coenzyme Q10+fatty acid metabolism inhibitor at the same Coenzyme Q10 concentration, **** indicates p<0.0001; and * indicates p<0.05. Statistical analysis was performed by two-way ANOVA.

FIG. 2A shows the effects of various concentrations of Coenzyme Q10 (CoQ10) alone or in combination with the fatty acid metabolism inhibitor Etomoxir (50 μM) on cell viability in MDA-MB231 (upper panel) or SKBr-3 (lower panel) breast cancer cells. FIGS. 2B and 2C show the effects of various concentrations of Coenzyme Q10 (CoQ10) alone or in combination with 100 μM Etomoxir (FIG. 2B) or 250 μM Etomoxir (FIG. 2C) on cell viability in SKBr-3 cells. **** indicates p<0.0001; and * indicates p<0.05. Statistical analysis was performed by unpaired T-test.

FIG. 3 shows the effects of various concentrations of Coenzyme Q10 (CoQ10) alone or in combination with the fatty acid metabolism inhibitor Trimetazidine (375 μM) on cell viability in MDA-MB231 (upper panel) or SKBr-3 (lower panel) breast cancer cells. **** indicates p<0.0001; and * indicates p<0.05. Statistical analysis was performed by unpaired T-test.

FIG. 4 shows the effects of various concentrations of Coenzyme Q10 (CoQ10) alone or in combination with metformin (500 μM) on cell viability in MDA-MB231 (upper panel) or SKBr-3 (lower panel) breast cancer cells.

FIG. 5 shows mRNA expression levels of the fatty acid metabolism genes FASN (upper left panel), CPT1 (lower left panel), ACSL1 (upper right panel) and DGAT1 (lower right panel) in MDA-MB231 breast cancer cells treated with Coenzyme Q10. *p<0.05, **p<0.01, ***p<0.001, ****p<0.00001, compared to control, N=3 biological replicates performed in triplicate ±SEM.

FIG. 6A shows free glycerol (24 hr exposure: upper panel; 48 hr exposure: lower panel) in MDA-MB231 breast cancer cells treated with Coenzyme Q10. FIGS. 6B and 6C show diacylglyceride (DAG) (B), and triacylglyceride (TAG) content (C) in MDA-MB231 breast cancer cells treated with Coenzyme Q10. FIG. 6D shows a cartoon of the structure of diacylglyceride and triacylglyceride. **p<0.01, ***p<0.001, ****p<0.00001, compared to untreated, N=6 biological replicates ±SEM.

FIG. 7A shows levels of various diacyl-glycerols (DAG) (upper panels) and triacyl-glycerols (TAG) (lower panels) in MDA-MB231 breast cancer cells treated with Coenzyme Q10. FIG. 7B shows the amount of Coenzyme Q10 in MDA-MB231 cells treated with Coenzyme Q10. FIG. 7C shows the ratio of Coenzyme Q10 to endogenous Coenzyme Q9 in MDA-MB231 cells treated with Coenzyme Q10. * p<0.05, **p<0.01 ***p<0.001, ****p<0.0001 compared to untreated, N=6 biological replicates ±SEM.

FIG. 8 shows the effects of various concentrations of Coenzyme Q10 alone or in combination with various concentrations of metformin (0.5, 1 and 5 mM) on cell viability in MIA PaCa-2 pancreatic cancer cells.

FIG. 9 shows the effects of various concentrations of Coenzyme Q10 alone or in combination with Etomoxir (100 μM), Trimetazidine (250 μM) or C75 (10μ) on cell viability in MIA PaCa-2 pancreatic cancer cells.

FIG. 10 shows the effects of various concentrations of Coenzyme Q10 alone or in combination with the SCD-1 inhibitor A939572 on cell viability in MIA PaCa-2 pancreatic cancer cells.

### DETAILED DESCRIPTION

### I. Definitions

In accordance with the present disclosure and as used herein, the following terms are defined with the following meanings, unless explicitly stated otherwise.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, “continuous infusion” is understood as administration of a therapeutic agent continuously for a period of at least 24, 48, 72 or 96 hours. Continuous infusion is typically accomplished by the use of a pump, optionally an implantable pump. A continuous infusion may be administered within the context of a treatment cycle. For example, a dose of a therapeutic agent can be administered by continuous infusion over a 24, 48, 72 or 96 hour period once per week each week. Treatment with continuous infusion does not require infusion of the therapeutic agent to the subject for the entire treatment period.

It is understood that continuous infusion can include short interruptions of administration, for example, to change the reservoir of coenzyme Q10 being administered. Continuous administration is typically facilitated by the use of a pump. Continuous infusion is carried out without including any significant interruptions of dosing by design. As used herein, interruptions to assess vital signs and/or perform laboratory assessments to ensure the safety of the patients and that no unacceptable adverse event have occurred are not considered to be significant interruptions. Interruptions resulting from equipment failure, e.g., pump failure, are not interruptions by design.

The terms “oncological disorder”, “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features.

As used herein, “oncological disorder”, “cancer” or “tumor” refers to all types of cancer or neoplasm or malignant tumors found in humans, including, but not limited to: leukemias, lymphomas, melanomas, carcinomas and sarcomas. As used herein, the terms or language “oncological disorder”, “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells.

A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

As used herein, a “detectable tumor” is a tumor that can be confirmed to be present in a subject, for example, using imaging methods (e.g., x-ray, CT scan, magnetic resonance imaging either with or without contrast agents, ultrasound), palpation or other physical examination methods, and/or direct observation by surgical methods or biopsy, typically coupled with histological analysis, in the case of a solid tumors; or by analysis of blood samples, e.g., completely blood count or histological analysis in the case of non-solid tumors, e.g., leukemias. In certain embodiments, a tumor can be detected based on the presence or certain markers. It is understood that diagnosis and detection of a tumor may involve multiple tests and diagnostic methods.

The term “sarcoma” generally refers to a tumor which is made up of a substance like the embryonic connective tissue and is generally composed of closely packed cells embedded in a fibrillar or homogeneous substance. Examples of sarcomas which can be treated using the methods of the invention include, for example, a chondrosarcoma, fibro sarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated using the methods of the invention include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated using the methods of the invention include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, merkel cell carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Specific criteria for the staging of cancer are dependent on the specific cancer type based on tumor size, histological characteristics, tumor markers, and other criteria known by those of skill in the art. Generally, cancer stages can be described as follows:

Stage 0 Carcinoma in situ

Stage I, Stage II, and Stage III Higher numbers indicate more extensive disease: Larger tumor size and/or spread of the cancer beyond the organ in which it first developed to nearby lymph nodes and/or tissues or organs adjacent to the location of the primary tumor

Stage IV The cancer has spread to distant tissues or organs

As used herein, the terms “treat,” “treating” or “treatment” refer, preferably, to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) state of disease, amelioration or palliation of the disease state, diminishing rate of or time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, treatment does not include one or more of a decrease in pain or an increase in the quality of life (QOL) as judged by a qualified individual, e.g., a treating physician, e.g., using accepted assessment tools of pain and QOL.

RECIST criteria are clinically accepted assessment criteria used to provide a standard approach to solid tumor measurement and provide definitions for objective assessment of change in tumor size for use in clinical trials. Such criteria can also be used to monitor response of an individual undergoing treatment for a solid tumor. The RECIST 1.1 criteria are discussed in detail in Eisenhauer et al., New response evaluation criteria in solid tumors: Revised RECIST guideline (version 1.1). Eur. J. Cancer. 45:228-247, 2009, which is incorporated herein by reference. Response criteria for target lesions include:

Complete Response (CR): Disappearance of all target lesions. Any pathological lymph nodes (whether target or non-target) must have a reduction in short axis to <10 mm.

Partial Response (PR): At least a 30% decrease in the sum of diameters of target lesion, taking as a reference the baseline sum diameters.

Progressive Diseases (PD): At least a 20% increase in the sum of diameters of target lesions, taking as a reference the smallest sum on the study (this includes the baseline sum if that is the smallest on the study). In addition to the relative increase of 20%, the sum must also demonstrate an absolute increase of at least 5 mm. (Note: the appearance of one or more new lesions is also considered progression.)

Stable Disease (SD): Neither sufficient shrinkage to qualify for PR nor sufficient increase to qualify for PD, taking as a reference the smallest sum diameters while on study.

RECIST 1.1 criteria also consider non-target lesions which are defined as lesions that may be measureable, but need not be measured, and should only be assessed qualitatively at the desired time points. Response criteria for non-target lesions include:

Complete Response (CR): Disappearance of all non-target lesions and normalization of tumor marker levels. All lymph nodes must be non-pathological in size (<10 mm short axis).

Non-CR/Non-PD: Persistence of one or more non-target lesion(s) and/ or maintenance of tumor marker level above the normal limits.

Progressive Disease (PD): Unequivocal progression (emphasis in original) of existing non-target lesions. The appearance of one or more new lesions is also considered progression. To achieve “unequivocal progression” on the basis of non-target disease, there must be an overall level of substantial worsening of non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR in target disease will therefore be extremely rare.

Clinically acceptable criteria for response to treatment in acute leukemias are as follows:

Complete remission (CR): The patient must be free of all symptoms related to leukemia and have an absolute neutrophil count of ≥1.0×10⁹/L, platelet count ≥100×10⁹/L, and normal bone marrow with <5% blasts and no Auer rods.

Complete remission with incomplete blood count recovery (Cri): As per CE, but with residual thrombocytopenia (platelet count <100×10⁹/L) or residual neutropenia (absolute neutrophil count <1.0×10⁹/L).

Partial remission (PR): A ≥50% decrease in bone marrow blasts to 5 to 25% abnormal cells in the marrow; or CR with ≤5% blasts if Auer rods are present.

Treatment failure: Treatment has failed to achieve CR, Cri, or PR. Recurrence.

Relapse after confirmed CR: Reappearance of leukemic blasts in peripheral blood or ≥5% blasts in the bone marrow not attributable to any other cause (e.g., bone marrow regeneration after consolidated therapy) or appearance of new dysplastic changes.

In certain embodiments, the Coenzyme Q10 and at least one fatty acid metabolism inhibitor are administered to the subject with one or more additional chemotherapeutic agents.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

A “chemotherapeutic regimen” is a clinically accepted dosing protocol for the treatment of cancer that includes administration of one or more chemotherapeutic agents to a subject in specific amounts on a specific schedule. In certain embodiments, the chemotherapeutic agent can be an agent in clinical trials.

As used herein, “co-administration” or “combination therapy” is understood as administration of two or more active agents using separate formulations or a single pharmaceutical formulation, or consecutive administration in any order such that, there is a time period while both (or all) active agents simultaneously exert their biological activities. It is contemplated herein that one active agent (e.g., CoQ10) can improve the activity of a second agent (e.g. a fatty acid metabolism inhibitor), for example, can sensitize target cells, e.g., cancer cells, to the activities of the second agent. Co-administration does not require that the agents are administered at the same time, at the same frequency, or by the same route of administration. As used herein, “co-administration” or “combination therapy” includes administration of a CoQ10 compound with one or more fatty acid metabolism inhibitors, or administration of two or more CoQ10 compounds. Examples of fatty acid metabolism inhibitors are provided herein.

Treatment regimens can include administration of a drug on a predetermined “cycle” including intervals of dosing and not dosing with one or more agents for the treatment of cancer. For example, an agent can be administered one or more times per week for three consecutive weeks followed by a week of no agent administered to provide a four week cycle. The cycle can be repeated so that the subject would be subjected to three treatment weeks, one no treatment week, three treatment weeks, one no treatment week, etc., for the desired number of cycles. In certain embodiments, treatment of efficacy and laboratory values (e.g., liver enzymes, blood count, kidney function) are assessed at the end of each cycle or every other cycle.

A “subject who has failed a chemotherapeutic regimen” is a subject with cancer that does not respond, or ceases to respond to treatment with a chemotherapeutic regimen per RECIST 1.1 criteria (see, Eisenhauer et al., 2009 and as discussed above), i.e., does not achieve at least stable disease (i.e., stable disease, partial response, or complete response) in the target lesion; or does not achieve at least non-CR/non-PD (i.e., non-CR/non-PD or complete response) of non-target lesions, either during or after completion of the chemotherapeutic regimen, either alone or in conjunction with surgery and/or radiation therapy which, when possible, are often clinically indicated in conjunction with chemotherapy. A failed chemotherapeutic regime results in, e.g., tumor growth, increased tumor burden, and/ or tumor metastasis. In some embodiments, failed chemotherapeutic regimen as used herein includes a treatment regimen that was terminated due to a dose limiting toxicity, e.g., a grade III or a grade IV toxicity that cannot be resolved to allow continuation or resumption of treatment with the fatty acid metabolism inhibitor or regimen that caused the toxicity. In some embodiments, a “failed chemotherapeutic regimen includes a treatment regimen that does not result in at least stable disease for all target and non-target lesions for an extended period, e.g., at least 1 month, at least 2 months, at least 3 months, at least 4 months, at least 5 months, at least 6 months, at least 12 months, at least 18 months, or any time period less than a clinically defined cure. In some embodiments, a failed chemotherapeutic regimen includes a treatment regimen that results in progressive disease of at least one target lesion during treatment with the fatty acid metabolism inhibitor, or results in progressive disease less than 2 weeks, less than 1 month, less than two months, less than 3 months, less than 4 months, less than 5 months, less than 6 months, less than 12 months, or less than 18 months after the conclusion of the treatment regimen, or less than any time period less than a clinically defined cure.

A failed chemotherapeutic regimen does not include a treatment regimen wherein the subject treated for a cancer achieves a clinically defined cure, e.g., 5 years of complete response after the end of the treatment regimen, and wherein the subject is subsequently diagnosed with a distinct cancer, e.g., more than 5 years, more than 6 years, more than 7 years, more than 8 years, more than 9 years, more than 10 years, more than 11 years, more than 12 years, more than 13 years, more than 14 years, or more than 15 years after the end of the treatment regimen. For example, a subject who suffered from a pediatric cancer may develop cancer later in life after being cured of the pediatric cancer. In such a subject, the chemotherapeutic regimen to treat the pediatric cancer is considered to have been successful.

A “refractory cancer” is a malignancy for which surgery is ineffective, which is either initially unresponsive to chemo- or radiation therapy, or which becomes unresponsive to chemo- or radiation therapy over time.

The terms “administer”, “administering” or “administration” include any method of delivery of a pharmaceutical composition or agent into a subject's system or to a particular region in or on a subject. In certain embodiments, the agent is delivered orally. In certain embodiments, the agent is administered parenterally. In certain embodiments, the agent is delivered by injection or infusion. In certain embodiments, the agent is delivered topically including transmucosally. In certain embodiments, the agent is delivered by inhalation. In certain embodiments of the invention, an agent is administered by parenteral delivery, including, intravenous, intramuscular, subcutaneous, intramedullary injections, as well as intrathecal, direct intraventricular, intraperitoneal, intranasal, or intraocular injections. In one embodiment, the compositions provided herein may be administered by injecting directly to a tumor. In some embodiments, the formulations of the invention may be administered by intravenous injection or intravenous infusion. In certain embodiments, the formulation of the invention can be administered by continuous infusion. In certain embodiments, administration is not oral. In certain embodiments, administration is systemic. In certain embodiments, administration is local. In some embodiments, one or more routes of administration may be combined, such as, for example, intravenous and intratumoral, or intravenous and peroral, or intravenous and oral, intravenous and topical, or intravenous and transdermal or transmucosal. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

“Adverse events” or “AEs” are characterized by grade depending on the severity. Some AE (e.g., nausea, low blood counts, pain, reduced blood clotting) can be treated so that the specific chemotherapeutic regimen can be continued or resumed. Some adverse events (e.g., loss of cardiac, liver, or kidney function; nausea) may not be treatable, requiring termination of treatment with the drug. Determination of AE grade and appropriate interventions can be determined by those of skill in the art. Common Terminology Criteria for Adverse Events v4.0 (CTCAE) (Publish Date: May 28, 2009) provide a grading scale for adverse events as follows:

Grade 1 Mild; asymptomatic or mild symptoms; clinical or diagnostic observations only; intervention not indicated.

Grade 2 Moderate; minimal, local or noninvasive intervention indicated; limiting age-appropriate instrumental activities of daily life (ADL).

Grade 3 Severe or medically significant but not immediately life-threatening; hospitalization or prolongation of hospitalization indicated; disabling, limiting self care ADL.

Grade 4 Life-threatening consequences; urgent intervention indicated.

Grade 5 Death related to adverse event.

As used herein, the term “survival” refers to the continuation of life of a subject which has been treated for a disease or condition, e.g., cancer. The time of survival can be defined from an arbitrary point such as time of entry into a clinical trial, time from completion or failure or an earlier treatment regimen, time from diagnosis, etc.

As used herein, “opsonization” refers to the process by which a lipophilic bioactive agent as described herein is marked for ingestion and destruction by a phagocyte. Opsonization involves the binding of an opsonin to bioactive agent. After opsonin binds to the membrane, phagocytes are attracted to the active agent. An opsonin is any molecule that acts as a binding enhancer for the process of phagocytosis.

As used herein, the term “opsonization reducer” refers to any agent that works in conjunction with the active agent to reduce the ability of opsonins to act as a binding enhancer for the process of phagocytosis.

As used herein, a “dispersion” refers to a system in which particles of colloidal size of any nature (e.g., solid, liquid or gas) are dispersed in a continuous phase of a different composition or state. In intravenous drug delivery the continuous phase is substantially water and the dispersed particles can be solid (a suspension) or an immiscible liquid (emulsion).

A “subject” to be treated by the method of the invention can mean either a human or non-human animal, preferably a mammal, more preferably a human. In certain embodiments, a subject has a detectable tumor prior to initiation of treatments using the methods of the invention. In certain embodiments, the subject has a detectable tumor at the time of initiation of the treatments using the methods of the invention.

As used herein, the term “safe and therapeutic effective amount” refers to the quantity of a component which is sufficient to yield a desired therapeutic response without undue adverse side effects (such as toxicity, irritation, or allergic response) commensurate with a reasonable benefit/risk ratio when used in the manner of this disclosure.

“Therapeutically effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” will vary depending on the compound, the disease and its severity and the age, weight, etc., of the patient to be treated. A therapeutically effective amount need not be curative. A therapeutically effective amount need not prevent a disease or condition from ever occurring. Instead a therapeutically effective amount is an amount that will at least delay or reduce the onset, severity, or progression of a disease or condition. Disease progression can be monitored, for example, by one or more of tumor burden, time to progression, survival time, or other clinical measurements used in the art.

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like.

“Preventing” or “prevention” refers to a reduction in risk of acquiring a disease or disorder (i.e., causing at least one of the clinical signs or symptoms of the disease not to develop in a patient that may be exposed to or predisposed to the disease but does not yet experience or display symptoms of the disease). Prevention does not require that the disease or condition never occur, or recur, in the subject.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

In all occurrences in this application where there are a series of recited numerical values, it is to be understood that any of the recited numerical values may be the upper limit or lower limit of a numerical range. It is to be further understood that the invention encompasses all such numerical ranges, i.e., a range having a combination of an upper numerical limit and a lower numerical limit, wherein the numerical value for each of the upper limit and the lower limit can be any numerical value recited herein. Ranges provided herein are understood to include all values within the range. For example, 1-10 is understood to include all of the values 1, 2, 3, 4, 5, 6, 7, 8, 9, and 10, and fractional values as appropriate. Ranges expressed as “up to” a certain value, e.g., up to 5, is understood as all values, including the upper limit of the range, e.g., 0, 1, 2, 3, 4, and 5, and fractional values as appropriate. Up to or within a week is understood to include, 0.5, 1, 2, 3, 4, 5, 6, or 7 days. Similarly, ranges delimited by “at least” are understood to include the lower value provided and all higher numbers.

All percent formulations are w/w unless otherwise indicated.

As used herein, “about” is understood to include within three standard deviations of the mean or within standard ranges of tolerance in the specific art. In certain embodiments, about is understood a variation of no more than 0.5.

The term “including” is used herein to mean, and is used interchangeably with, the phrase “including but not limited to”.

The term “or” is used inclusively herein to mean, and is used interchangeably with, the term “and/or,” unless context clearly indicates otherwise.

The term “such as” is used herein to mean, and is used interchangeably, with the phrase “such as but not limited to”.

The term “standard dosage” as used herein refers to a dosage of a therapeutic agent that is commonly used for treatment of a disorder. For example, the recommended dosage of a therapeutic agent described in a product insert by a manufacturer of the therapeutic agent would be considered a standard dosage.

II. Coenzyme 010 Compounds

It will be understood that all of the methods provided in the instant invention may involve administration of, in place of Coenzyme Q10, any other Coenzyme Q10 compound, or a combination thereof. Coenzyme Q10 compounds are intended to include a class of CoQ10 compounds. Coenzyme Q10 compounds effective for the methods described herein include CoQ10, a metabolite of CoQ10, a biosynthetic precursor of CoQ10, an analog of CoQ10, a derivative of CoQ10, and CoQ10 related compounds. An analog of CoQ10 includes analogs having no or at least one isoprenyl repeats. CoQ10 has the following structure:

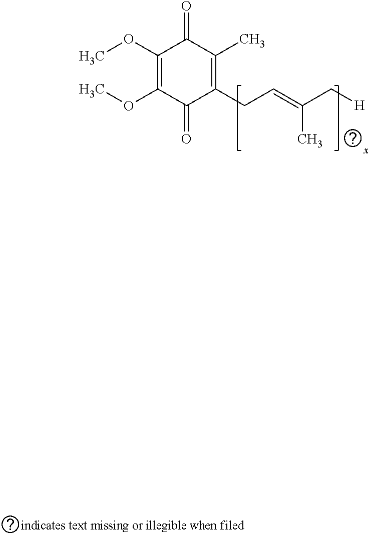

wherein x is 10. In the instant invention, CoQ10 compounds can include derivatives of CoQ10 in which x is any number of isoprenyl units from 4-10, or any number of isoprenyl units from 6-10, or any number of isoprenyl units from 8-10, or 9-10 isoprenyl units. CoQ10 includes the fully oxidized version, also known as ubiquinone, the partially oxidized version, also known as semiquinone or ubisemiquinone, or the fully reduced version, also known as ubiquinol; or any mixtures or combinations thereof. In certain embodiments, the CoQ10 compound for treatment of cancer is ubiquinone. In certain embodiments, the CoQ10 compound for treatment of cancer is ubiquinol.

In certain embodiments of the present invention, the therapeutic agent is Coenzyme Q10 (CoQ10). Coenzyme Q10, also referred to herein as CoQ10, is also known as ubiquinone, or ubidecarenone. CoQ10 is art-recognized and further described in International Publication No. WO 2005/069916 (Appln. No. PCT/US2005/001581), WO 2008/116135 (Appln. No. PCT/US08/57786), WO2010/132507 (Appln. No. PCT/US2010/034453), WO 2011/112900 (Appln. No. PCT/US2011/028042), and WO2012/174559 (Appln. No. PCT/US2012/043001) the entire contents of each of which are expressly incorporated by reference herein. CoQ10 is one of a series of polyprenyl 2,3-dimethoxy-5-methylbenzoquinone (ubiquinone) present in the mitochondrial electron transport systems of eukaryotic cells. Human cells produce CoQ10 exclusively and it is found in cell and mitochondrial membranes of all human cells, with the highest levels in organs with high energy requirements, such as the liver and the heart. The body pool of CoQ10 has been estimated to be about 2 grams, of which more than 50% is endogenous. Approximately 0.5 grams of CoQ10 is required from the diet or biosynthesis each day. CoQ10 is produced in ton quantities from the worldwide supplement market and can be obtained from Kaneka, with plants in Pasadena, Texas and Takasagoshi, Japan.

Coenzyme Q10 related compounds include, but are not limited to, benzoquinones, isoprenoids, farnesols, farnesyl acetate, farnesyl pyrophosphate, 1-phenylalanine, d-phenylalanine, dl-phenylalanine, 1-tyrosine, d-tyrosine, dl-tyrosine, 4-hydroxy-phenylpyruvate, 4-hydroxy-phenyllactate, 4-hydroxy-cinnamate, dipeptides and tripeptides of tyrosine or phenylalanine, 3,4-dihydroxymandelate, 3-methoxy-4-hydroxyphenylglycol, 3-methoxy-4-hydroxymandelate, vanillic acid, phenylacetate, pyridoxine, S-adenosyl methionine, panthenol, mevalonic acid, isopentyl pyrophosphate, phenylbutyrate, 4-hydroxy-benzoate, decaprenyl pyrophosphate, beta-hydroxybutyrate, 3-hydroxy-3-methyl-glutarate, acetylcarnitine, acetoacetylcarnitine, acetylglycine, acetoacetylglycine, carnitine, acetic acid, pyruvic acid, 3-hydroxy-3-methylglutarylcarnitine, all isomeric forms of serine, alanine, cysteine, glycine, threonine, hydroxyproline, lysine, isoleucine, and leucine, even carbon number C4 to C8 fatty acids (butyric, caproic, caprylic, capric, lauric, myristic, palmitic, and stearic acids) salts of carnitine and glycine, e.g., palmitoylcarnitine and palmitoylglycine, and 4-hydroxy-benzoate polyprenyltransferase, any salts of these compounds, as well as any combinations thereof, and the like. In certain embodiments, such agents can be used for the treatment of a cancer according to the methods provided herein.

Metabolites and biosynthetic precursors of CoQ10 include, but are not limited to, those compounds that are formed between the chemical/biological conversion of tyrosine and acetyl-CoA to ubiquinol. Intermediates of the coenzyme biosynthesis pathway include tyrosine, acetyl-CoA, 3-hexaprenyl-4-hydroxybenzoate, 3-hexaprenyl-4,5-dihydroxybenzoate, 3-hexaprenyl-4-hydroxy-5-methoxybenzoate, 2-hexaprenyl-6-methoxy-1,4-benzoquinone, 2-hexaprenyl-3-methyl-6-methoxy-1,4-benzoquinone, 2-hexaprenyl-3-methyl-5-hydroxy-6-methoxy-1,4-benzoquinone, 3-Octaprenyl-4-hydroxybenzoate, 2-octaprenylphenol, 2-octaprenyl-6-metholxyphenol, 2-octaprenyl-3-methyl-6-methoxy-1,4-benzoquinone, 2-octaprenyl-3-methyl-5-hydroxy-6-methoxy-1,4-benzoquinone, 2-decaprenyl-3-methyl-5-hydroxy-6-methoxy-1,4-benzoquinone, 2-decaprenyl-3-methyl-6-methoxy-1,4-benzoquinone, 2-decaprenyl-6-methoxy-1,4-benzoquinone, 2-decaprenyl-6-methoxyphenol, 3-decaprenyl-4-hydroxy-5-methoxybenzoate, 3-decaprenyl-4,5-dihydroxybenzoate, 3-decaprenyl-4-hydroxybenzoate, 4-hydroxy phenylpyruvate, 4-hydroxyphenyllactate, 4-hydroxy-benzoate, 4-hydroxycinnamate, and hexaprenydiphosphate. In certain embodiments, such agents can be used for the treatment of a cancer according to the methods provided herein.

### III. Compositions

The present disclosure provides compositions containing a CoQ10 compound, e.g., Coenzyme Q10, for use in a combination therapy for the treatment and prevention of cancer. The compositions of the present disclosure can be administered to a patient either by themselves, or in pharmaceutical compositions where it is mixed with suitable carriers or excipient(s). In treating a patient exhibiting an oncological disorder, a therapeutically effective amount of the CoQ10 compound is administered.

Suitable routes of administration of the present compositions of the invention may include parenteral delivery, including, intravenous, intramuscular, subcutaneous, intramedullary injections, as well as intrathecal, direct intraventricular, intraperitoneal, intranasal, or intraocular injections, just to name a few. In one embodiment, the compositions provided herein may be administered by injecting directly to a tumor. In some embodiments, the formulations of the invention may be administered by intravenous injection or intravenous infusion. In some embodiments, the formulation is administered by continuous infusion. In one embodiment, the compositions of the invention are administered by intravenous injection. In one embodiment, the compositions of the invention are administered by intravenous infusion. Where the route of administration is, for example intravenous infusion, embodiments are provided herein where the IV infusion comprises the active agent, e.g., CoQ10, at approximately a 40 mg/mL concentration. Where the composition is administered by IV infusion, it can be diluted in a pharmaceutically acceptable aqueous solution such as phosphate buffered saline or normal saline. In some embodiments, one or more routes of administration may be combined, such as, for example, intravenous and intratumoral, or intravenous and peroral, or intravenous and oral, or intravenous and topical, transdermal, or transmucosal.

The compositions described herein may be administered to a subject in any suitable formulation. These include, for example, liquid, semi-solid, and solid dosage forms, such as liquid solutions (e.g., injectable and infusible solutions), dispersions or suspensions, tablets, pills, powders, creams, lotions, liniments, ointments, or pastes, drops for administration to the eye, ear or nose, liposomes, and suppositories. The preferred form depends on the intended mode of administration and therapeutic application.

In certain embodiments, a CoQ10 compound, e.g., CoQ10, may be prepared with a carrier that will protect against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

For example, a CoQ10 compound e.g., CoQ10, can be formulated for parenteral delivery, e.g., for subcutaneous, intravenous, intramuscular, or intratumoral injection. The compositions may be administered in a single bolus, multiple injections, or by continuous infusion (for example, intravenously or by peritoneal dialysis). For parenteral administration, the compositions may be formulated in a sterilized pyrogen-free form.

Use of pharmaceutically acceptable carriers to formulate the compounds herein disclosed, for the practice of the present invention, into dosages suitable for systemic administration is within the scope of the present disclosure. With proper choice of carrier and suitable manufacturing practice, the compositions of the present disclosure, in particular, those formulated as solutions, may be administered parenterally, such as by intravenous injection.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices may be desirable. The data obtained from these cell culture assays and animal studies can be used in formulating a range of dosage for use in human. The dosage of such compounds may be within a range of circulating concentrations that include the ED50 with little or no toxicity. The dosage may vary within this range depending upon the dosage form employed and the route of administration utilized.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically. The preparations formulated for intravenous administration may be in the form of solutions of colloidal dispersion.

Pharmaceutical compositions for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

### IV. Formulations

The active agent, e.g., a CoQ10 compound, e.g., CoQ10, can be delivered in any pharmaceutically acceptable carrier for the desired route of administration. As used herein, formulations including CoQ10 compounds are formulated for any route of administration unless otherwise clearly indicated. In preferred embodiments, the formulations are for administration by injection, infusion, or topical administration. In certain embodiments, the CoQ10 compounds are not delivered orally.

Preferred therapeutic formulations for use in the methods of the invention comprise the active agent (e.g., a CoQ10 compound, e.g., CoQ10) in a microparticle formation, e.g., for intravenous administration. Such intravenous formulations are provided, for example, in WO2011/112900 (Appln. No. PCT/US2011/028042), the entire contents of which are expressly incorporated herein by reference, and an exemplary intravenous formulation as described in WO2011/112900 (Appln. No. PCT/US2011/028042). Through high pressure homogenization, active agent (e.g., a CoQ10 compound, e.g., CoQ10) particles are reduced to produce particles that are small enough to pass through a 200-nm sterilizing filter. Particles that are small enough to pass through a 200-nm sterilizing filter can be injected intravenously. These particles are much smaller than blood cells and therefore will not embolize capillaries. Red blood cells for example are 6-micron×2-micron disks. The particles are dispersed to and are encased or surrounded by a stabilizing agent. While not wishing to be bound by any theory, it is believed that the stabilizing agents are attracted to the hydrophobic therapeutic agent such that the dispersed particles of the hydrophobic therapeutic agent are surrounded by the stabilizing agent forming a suspension or an emulsion. The dispersed particles in the suspension or emulsion comprises a stabilizing agent surface and a core consisting of the hydrophobic therapeutic agent, e.g., a CoQ10 compound, e.g., CoQ10, in a solid particulate form (suspension) or in an immiscible liquid form (emulsion). The dispersed particles can be entrenched in the lipophilic regions of a liposome.

In certain embodiments the pharmaceutical composition of the invention is a lipid composition. The term “lipid composition” as used herein refers to a pharmaceutical composition comprising a lipid. Suitable lipids may include, but are not limited to fats, waxes, sterols, fat-soluble vitamins (such as vitamins A, D, E, and K), monoglycerides, diglycerides, triglycerides, phospholipids, fatty acids, glycerolipids, glycerophospholipids, sphingolipids, saccharolipids, polyketides (derived from condensation of ketoacyl subunits), sterol lipids and prenol lipids (derived from condensation of isoprene subunits). In a particular embodiment, the lipid composition comprises a phospholipid. Suitable phospholipids include, but are not limited to, lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, and combinations thereof.

Lipid compositions may also include an oil phase which, in turn, may include emollients, fatty alcohols, emulsifiers, combinations thereof, and the like. For example, an oil phase could include emollients such as C12-15 alkyl benzoates (commercially available as FINSOLV™ TN from Finetex Inc. (Edison, N.J.)), capric-caprylic triglycerides (commercially available from Huls as MIGLYOL™ 812), and the like. Other suitable emollients which may be utilized include vegetable derived oils (corn oil, safflower oil, olive oil, macadamian nut oil, etc.); various synthetic esters, including caprates, linoleates, dilinoleates, isostearates, fumarates, sebacates, lactates, citrates, stearates, palmitates, and the like; synthetic medium chain triglycerides, silicone oils or polymers; fatty alcohols such as cetyl alcohol, stearyl alcohol, cetearyl alcohol, lauryl alcohol, combinations thereof, and the like; and emulsifiers including glyceryl stearate, PEG-100 stearate, Glyceryl Stearate, Glyceryl Stearate SE, neutralized or partially neutralized fatty acids, including stearic, palmitic, oleic, and the like; vegetable oil extracts containing fatty acids, Ceteareth®-20, Ceteth®-20, PEG-150 Stearate, PEG-8 Laurate, PEG-8 Oleate, PEG-8 Stearate, PEG-20 Stearate, PEG-40 Stearate, PEG-150 Distearate, PEG-8 Distearate, combinations thereof, and the like; or other non-polar cosmetic or pharmaceutically acceptable materials used for skin emolliency within the purview of those skilled in the art, combinations thereof, and the like.

Lipid compositions can also include a liposomal concentrate including, for example, a phospholipid such as lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, and combinations thereof, at least one lipophilic bioactive agent, and at least one solubilizer. The phospholipid can also include DPPC, DSPC, DMPC, or a combination thereof. The liposomal concentrate may be in combination with at least one pharmaceutically acceptable carrier possessing at least one permeation enhancer in an amount from about 0.5% by weight to about 20% by weight of the composition. The phospholipid may present in the composition in an amount from about 2% to about 20% by weight of the composition and the bioactive agent may be present in an amount from about 0.5% to about 20% by weight of the composition.

In some embodiments, the lipid composition comprises a dispersion stabilizing agent. In some embodiments, the dispersion stabilizing agent is selected from natural or semisynthetic phospholipids. For example, suitable stabilizing agents include Polyethoxylated (a/k/a pegylated) castor oil (Cremophor® EL), Polyethoxylated hydrogenated castor oil (Cremophor® RH 40), Tocopherol polyethylene glycol succinate (Pegylated vitamin E, Vitamin E TPGS), Sorbitan fatty acid esters (Spans®), Bile acids and bile-acid salts or Dimyristoylphosphatidyl choline (DMPC). In some embodiments the stabilizing agent is DMPC.

In some embodiments, the lipid composition may includes a dispersion of liposomal particles having an average diameter between about 30 and 500 nm, each liposomal particle comprising a hydrophobic bioactive agent, a phospholipid, and an aqueous dispersion vehicle. The ratio of hydrophobic bioactive agent:phospholipid is between about 5:1 and about 1:5, the hydrophobic bioactive agent is between about 0.1 and 30% w/w of the composition, the phospholipid is between about 0.1 and 30% w/w of the composition, and the liposomal particles are dispersed within the aqueous dispersion vehicle.

In certain embodiments, the lipid is at least 10, 9, 8, 7, 6, 5, 4, 3, 2, or 1% w/w of the composition. In certain embodiments, the lipid is less than 10, 9, 8, 7, 6, 5, 4, 3, 2, or 1% w/w of the composition. In certain embodiments, the lipid is about 10, 9, 8, 7, 6, 5, 4, 3, 2, or 1% w/w of the composition. Any of these values may be used to define a range for the lipid content of the lipid compositions. For example, in some embodiments, the lipid composition comprises 1% to 5%, 1% to 10%, or 1% to 4% w/w of the composition.

In some embodiments, the formulation is a dispersed colloidal system. Dispersed colloidal systems permit a high drug load in the formulation without the use of co-solvents. Additionally, high and relatively reproducible plasma levels are achieved without the dependence on endogenous low-density lipoprotein carriers. More importantly, the formulations allow sustained high drug levels in solid tumors due to the passive accumulation of the colloidal particles of the hydrophobic therapeutic agent.

A preferred intravenous formulation substantially comprises a continuous phase of water and dispersed solids (suspension) or dispersed immiscible liquid (emulsion). Dispersed colloidal systems, in which the particles are composed largely of the active agent (drug) itself, can often deliver more drug per unit volume than continuous solubilizing systems, if the system can be made adequately stable.

As the formulation medium, the aqueous solution may include Hank's solution, Ringer's solution, phosphate buffered saline (PB S), physiological saline buffer or other suitable salts or combinations to achieve the appropriate pH and osmolarity for parenterally delivered formulations. Aqueous solutions can be used to dilute the formulations for administration to the desired concentration. For example, aqueous solutions can be used to dilute a formulation for intravenous administration from a concentration of about 4% w/v to a lower concentration to facilitate administration of lower doses of CoQ10. The aqueous solution may contain substances which increase the viscosity of the solution, such as sodium carboxymethyl cellulose, sorbitol, or dextran.

The active agent (e.g., a CoQ10 compound, e.g., CoQ10) is dispersed in the aqueous solution such that a colloidal dispersion is formed wherein the nano-dispersion particles of the hydrophobic therapeutic agent are covered or encased or encircled by the dispersion stabilizing agents to form nano-dispersions of the active agent (e.g., a CoQ10 compound, e.g., CoQ10) particles. The nano-dispersed active agent (e.g., a CoQ10 compound, e.g., CoQ10) particles have a core formed of the hydrophobic therapeutic agent that is surrounded by the stabilizing agent. Similarly, in certain aspects, the stabilizing agent is a phospholipid having both a hydrophilic and lipophilic portion. The phospholipids form liposomes or other nanoparticles upon homogenization. In certain aspects these liposomes are bi-layered unilamellar liposomes while in other embodiments the liposomes are bi-layered multi-lamellar liposomes. The dispersed active agent (e.g., a CoQ10 compound, e.g., CoQ10) particles are dispersed in the lipophilic portion of the bi-layered structure of the liposome formed from the phospholipids. In certain other aspects the core of the liposome, like the core of the nano-dispersion of active agent (e.g., a CoQ10 compound, e.g., CoQ10) particles, is formed of the hydrophobic therapeutic agent and the outer layer is formed of the bi-layered structure of the phospholipid. In certain embodiments the colloidal dispersions are treated by a lyophilization process whereby the nanoparticle dispersion is converted to a dry powder.

In some embodiments, the formulation for injection or infusion used is a 4% sterile aqueous colloidal dispersion containing CoQ10 in a nanosuspension as prepared in WO2011/112900, the entire contents of which are incorporated herein by reference. In certain embodiments, the formulation includes an aqueous solution; a hydrophobic active agent, e.g., CoQ10, a CoQ10 precursor or metabolite or a CoQ10 related compound, dispersed to form a colloidal nano-dispersion of particles; and at least one of a dispersion stabilizing agent and an opsonization reducer; wherein the colloidal nano-dispersion of the active agent is dispersed into nano-dispersion particles having a mean size of less than 200-nm.

In certain embodiments, the dispersion stabilizing agent includes, but is not limited to, pegylated castor oil, Cremphor® EL, Cremophor® RH 40, Pegylated vitamin E, Vitamin E TPGS, and Dimyristoylphosphatidyl choline (DMPC).

In certain embodiments, the opsonization reducer is a poloxamer or a poloxamines.

In certain embodiments, the colloidal nano-dispersion is a suspension or an emulsion. Optionally, a colloidal nano-dispersion is in a crystalline form or a super-cooled melt form.

In certain embodiments, the formulation for injection or infusion includes a lyoprotectant such as a nutritive sugar including, but not limited to, lactose, mannose, maltose, galactose, fructose, sorbose, raffinose, neuraminic acid, glucosamine, galactosamine, N-methylglucosamine, mannitol, sorbitol, arginine, glycine and sucrose, or any combination thereof.

In certain embodiments, the formulation for injection or infusion includes an aqueous solution; a hydrophobic active agent dispersed to form a colloidal nano-dispersion of particles; and at least one of a dispersion stabilizing agent and an opsonization reducer. The colloidal nano-dispersion of the active agent is dispersed into nano-dispersion particles having sizes of less than 200 nm. In some embodiments the dispersion stabilizing agent is selected from natural or semisynthetic phospholipids. For example, suitable stabilizing agents include polyethoxylated (a/k/a pegylated) castor oil (Cremophor® EL), polyethoxylated hydrogenated castor oil (Cremophor® RH 40), Tocopherol polyethylene glycol succinate (Pegylated vitamin E, Vitamin E TPGS), Sorbitan fatty acid esters (Spans®), Bile acids and bile-acid salts or Dimyristoylphosphatidyl choline (DMPC). In some embodiments the stabilizing agent is DMPC.

In certain embodiments the formulation is suitable for parenteral administration, including intravenous, intraperitoneal, orthotopical, intracranial, intramuscular, subcutaneous, intramedullary injections, as well as intrathecal, direct intraventricular, intranasal, or intraocular injections. In certain embodiments, the formulation contains CoQ10, dimyristoyl-phophatidylcholine, and poloxamer 188 in a ratio of 4:3:1.5 respectively that is designed to stabilize the nanosuspension of the particles. In some embodiments, the formulation includes a phosphate buffer saline solution which contains sodium phosphate dibasic, potassium phosphate monobasic, potassium chloride, sodium chloride and water for injection. In certain embodiments, the 4% sterile aqueous colloidal dispersion containing CoQ10 in a nanosuspension is diluted in the phosphate buffered saline solution provided, e.g., 1:1, 1:2, 1:3, 1:4. 1:5, 1:6, 1:7, 1:8. 1:9, 1:10, 1:11, 1:12, 1:13, 1:14. 1:15, 1:16, 1:17, 1:18. 1:19, 1:20, or other appropriate ratio bracketed by any two of the values.

In some embodiments, the formulation is a topical formulation. Topical formulations of CoQ10 compounds are provided, for example in WO2010/132507 (PCT Appln. No. PCT/US2010/034453), WO2008116135 (PCT Appln. No. PCT/US2008/116135), and WO2005/069916 (PCT Appln. PC/US2005/001581), the entire contents of each of which are expressly incorporated herein by reference.

Formulations suitable for topical administration include liquid or semi-liquid preparations suitable for penetration through the skin, such as liniments, lotions, creams, ointments or pastes, and drops suitable for administration to the eye, ear, or nose. Drops according to the present disclosure may include sterile aqueous or oily solutions or suspensions and may be prepared by dissolving the active ingredient in a suitable aqueous solution of a bactericidal and/or fungicidal agent and/or any other suitable preservative, and in some embodiments including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present disclosure include those suitable for application to the skin or eye. An eye lotion may include a sterile aqueous solution optionally containing a bactericide and may be prepared by methods similar to those for the preparation of drops. Lotions or liniments for application to the skin may also include an agent to hasten drying and to cool the skin, such as an alcohol, and/or a moisturizer such as glycerol or an oil such as castor oil or arachis oil.

Creams, ointments or pastes useful in the methods of the invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may include hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

In some embodiments, the remaining component of a topical delivery vehicle may be water or a water phase, in embodiments purified, e.g. deionized, water, glycerine, propylene glycol, ethoxydiglycol, phenoxyethanol, and cross linked acrylic acid polymers. Such delivery vehicle compositions may contain water or a water phase in an amount of from about 50 to about 95 percent, based on the total weight of the composition. The specific amount of water present is not critical, however, being adjustable to obtain the desired viscosity (usually about 50 cps to about 10,000 cps) and/or concentration of the other components. The topical delivery vehicle may have a viscosity of at least about 30 centipoises.

Transdermal skin penetration enhancers can also be used to facilitate delivery of CoQ10. Illustrative are sulfoxides such as ethoxydiglycol, 1,3-butylene glycol, isopentyl diol, 1,2-pentane diol, propylene glycol, 2-methyl propan-2-ol, propan-2-ol, ethyl-2-hydroxypropanoate, hexan-2,5-diol, di(2-hydroxypropyl)ether, pentan-2,4-diol, acetone, polyoxyethylene(2)methyl ether, 2-hydroxypropionic acid, 2-hydroxyoctanoic acid, propan-1-ol, 1,4 dioxane, tetrahydrofuran, butan-1,4-diol, propylene glycol dipelargonate, polyoxypropylene 15 stearyl ether, octyl alcohol, polyoxyethylene ester of oleyl alcohol, oleyl alcohol, lauryl alcohol, dioctyl adipate, dicapryl adipate, diisopropyl adipate, diisopropyl sebacate, dibutyl sebacate, diethyl sebacate, dimethyl sebacate, dioctyl sebacate, dibuyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, isopropyl palmitate, ethyl laurate, 2-ethyl-hexyl pelargonate, isopropyl isostearate, butyl laurate, benzyl benzoate, butyl benzoate, hexyl laurate, ethyl caprate, ethyl caprylate, butyl stearate, benzyl salicylate, 2-hyroxyoctanoic acid, dimethyl sulphoxide, methyl sufonyl methane, n,n-dimethyl acetamide, n,n-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfural alcohol, urea, diethyl-m-toluamide, 1-dodecylazacyloheptan-2-one, and combinations thereof.

Solubilizers, particularly for topical administration can include, but are not limited to, polyoxyalkylene dextrans, fatty acid esters of saccharose, fatty alcohol ethers of oligoglucosides, fatty acid esters of glycerol, fatty acid esters of polyoxyethylenes, polyethoxylated fatty acid esters of sorbitan, fatty acid esters of poly(ethylene oxide), fatty alcohol ethers of poly(ethylene oxide), alkylphenol ethers of poly(ethylene oxide), polyoxyethylene-polyoxypropylene block copolymers, ethoxylated oils, and combinations thereof.

Topical formulations can include emollients, including, but not limited to, C12-15 alkyl benzoates, capric-caprylic triglycerides, vegetable derived oils, caprates, linoleates, dilinoleates, isostearates, fumarates, sebacates, lactates, citrates, stearates, palmitates, synthetic medium chain triglycerides, silicone oils, polymers and combinations thereof; the fatty alcohol is selected from the group consisting of cetyl alcohol, stearyl alcohol, cetearyl alcohol, lauryl alcohol and combinations thereof; and the emulsifier is selected from the group consisting of glyceryl stearate, polyethylene glycol 100 stearate, neutralized fatty acids, partially neutralized fatty acids, polyethylene glycol 150 stearate, polyethylene glycol 8 laurate, polyethylene glycol oleate, polyethylene glycol 8 stearate, polyethylene glycol 20 stearate, polyethylene glycol 40 stearate, polyethylene glycol 150 distearate, polyethylene glycol 8 distearate, and combinations thereof.

Topical formulations can include a neutralization phase comprising one or more of water, amines, sodium lactate, and lactic acid.

The water phase can further optionally include one or more of water phase comprises the permeation enhancer optionally in combination with a viscosity modifier selected from the group consisting of cross linked acrylic acid polymers, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed, rice starch, corn starch, potato starch, wheat starch, algae extract, dextran, succinoglucan, carboxymethyl starch, methylhydroxypropyl starch, sodium alginate, alginic acid propylene glycol esters, sodium polyacrylate, polyethylacrylate, polyacrylamide, polyethyleneimine, bentonite, aluminum magnesium silicate, laponite, hectonite, and anhydrous silicic acid.

Topical formulations can also include a pigment such as titanium dioxide.

In an embodiment, a topical formulation for use in the methods of the invention includes an oil phase comprising C12-15 alkyl benzoates or capric/caprylic triglyceride, cetyl alcohol, stearyl alcohol, glyceryl stearate, and polyethylene glycol 100 stearate, in an amount of from about 5% to about 20% by weight of the composition; a water phase comprising glycerin, propylene glycol, ethoxydiglycol, phenoxyethanol, water, and a crosslinked acrylic acid polymer, in an amount of from about 60 to about 80% by weight of the composition; a neutralization phase comprising water, triethanolamine, sodium lactate, and lactic acid, in an amount of from about 0.1% to about 15% by weight of the composition; a pigment comprising titanium dioxide in an amount of from about 0.2% to about 2% by weight of the composition; and a liposomal concentrate comprising a polyethoxylated fatty acid ester of sorbitan, coenzyme Q10, a phosphatidylcholine lecithin, phenoxyethanol, propylene glycol, and water, in an amount of from about 0.1% to about 30% by weight of the composition, wherein the propylene glycol and ethoxydiglycol are present in a combined amount of from 3% by weight to about 15% by weight of the composition and the coenzyme Q10 is present in an amount of from about 0.75% by weight to about 10% by weight of the composition. Other formulations for use in the methods of the invention are provided, for example, in WO2008/116135 (PCT Application No. PCT/US08/57786), and in WO2010/132507 (PCT/US2010/034453), the entire contents of each of which are expressly incorporated herein by reference.

In one embodiment, a topical formulation for use in the methods of the invention is a 3% CoQ10 cream as described in US 2011/0027247, the entire contents of which are incorporated by reference herein. In one embodiment, the 3% CoQ10 comprises:

(1) a phase A having C12-15 alkyl benzoate or capric/caprylic triglyceride at about 4.0% w/w of the composition, cetyl alcohol at about 2.00% w/w of the composition, stearyl alcohol at about 1.5% w/w, glyceryl stearate and PEG-100 at about 4.5% w/w;

(2) a phase B having glycerin at about 2.00% w/w, propylene glycol at about 1.5% w/w, ethoxydiglycol at about 5.0% w/w, phenoxyethanol at about 0.475% w/w, a carbomer dispersion at about 40% w/w, purified water at about 16.7% w/w;

(3) a phase C having triethanolamine at about 1.3% w/w, lactic acid at about 0.5% w/w, sodium lactate solution at about 2.0% w/w, water at about 2.5% w/w;

(4) a phase D having titanium dioxide at about 1.0% w/w; and

(5) a phase E having CoQ10 21% concentrate at about 15.0% w/w.

A CoQ10 21% concentrate composition (phase E in above 3% cream) can be prepared by combining phases A and B as described below. Phase A includes Ubidecarenone USP (CoQ10) at 21% w/w and polysorbate 80 NF at 25% w/w. Phase B includes propylene glycol USP at 10.00% w/w, phenoxyethanol NF at 0.50% w/w, lecithin NF (PHOSPHOLIPON 85G) at 8.00% w/w and purified water USP at 35.50% w/w. All weight percentages are relative to the weight of the entire CoQ10 21% concentrate composition. The percentages and further details are listed in the following table.

TABLE 1

| Phase | Trade Name | INCI Name | Percent |
|---|---|---|---|
| A | RITABATE 80 | POLYSORBATE 80 | 25.000 |
| A | UBIDECARENONE | UBIQUINONE | 21.000 |
| B | PURIFIED WATER | WATER | 35.500 |
| B | PROPYLENE GLYCOL | PROPYLENE | 10.000 |
| | | GLYCOL | |
| B | PHENOXYETHANOL | PHENOXYETHANOL | 0.500 |
| B | PHOSPHOLIPON 85G | LECITHIN | 8.000 |
| Totals | | | 100.000 |

The phenoxyethanol and propylene glycol are placed in a suitable container and mixed until clear. The required amount of water is added to a second container (Mix Tank 1). Mix Tank 1 is heated to between 45 and 55° C. while being mixed. The phenoxyethanol/propylene glycol solution is added to the water and mixed until it was clear and uniform. When the contents of the water phase in Mix Tank 1 are within the range of 45 to 55° C., Phospholipon G is added with low to moderate mixing. While avoiding any foaming, the contents of Mix Tank 1 is mixed until the Phospholipon 85G was uniformly dispersed. The polysorbate 89 is added to a suitable container (Mix Tank 2) and heated to between 50 and 60° C. The Ubidecarenone is then added to Mix Tank 2. While maintaining the temperature at between 50 and 60° C. Mix Tank 2 is mixed until all the Ubidecarenone is dissolved. After all the Ubidecarenone has been dissolved, the water phase is slowly transferred to Mix Tank 2. When all materials have been combined, the contents are homogenized until dispersion is smooth and uniform. While being careful not to overheat, the temperature is maintained at between 50 and 60° C. The homogenization is then stopped and the contents of Mix Tank 2 are transferred to a suitable container for storage.

In some embodiments, a formulation for any route of administration for use in the invention may include from about 0.001% to about 20% (w/w) of CoQ10, more preferably between about 0.01% and about 15% and even more preferably between about 0.1% to about 10% (w/w) of CoQ10. In certain embodiments, a formulation for any route of administration for use in the invention may include from about 1% to about 10% (w/w) of CoQ10. In certain embodiments, a formulation for any route of administration for use in the invention may include from about 2% to about 8% (w/w) of CoQ10. In certain embodiments, a formulation for any route of administration for use in the invention may include from about 2% to about 7% (w/w) of CoQ10. In certain embodiments, a formulation for any route of administration for use in the invention may include from about 3% to about 6% (w/w) of CoQ10. In certain embodiments, a formulation for any route of administration for use in the invention may include from about 3% to about 5% (w/w) of CoQ10. In certain embodiments, a formulation for any route of administration for use in the invention may include from about 3.5% to about 4.5% (w/w) of CoQ10. In certain embodiments, a formulation for any route of administration for use in the invention may include from about 3.5% to about 5% (w/w) of CoQ10. In one embodiment a formulation includes about 4% (w/w) of CoQ10. In one embodiment a formulation includes about 8% (w/w) of CoQ10. In various embodiments, the formulation includes about 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%, 17%, 18%, 19% or 20% (w/w) of CoQ10, or any range bracketed by any two values recited. In certain embodiments, the formulations can be prepared as a percent weight to volume rather than a percent weight to weight. Depending on the formulation, the concentration of CoQ10 may be the same, or about the same in the w/w and the w/v percent formulations. CoQ10 can be obtained from Kaneka Q10 as Kaneka Q10 (USP UBIDECARENONE) in powdered form (Pasadena, Texas, USA). CoQ10 used in the methods exemplified herein have the following characteristics: residual solvents meet USP 467 requirement; water content is less than 0.0%, less than 0.05% or less than 0.2%; residue on ignition is 0.0%, less than 0.05%, or less than 0.2% less than; heavy metal content is less than 0.002%, or less than 0.001%; purity of between 98-100% or 99.9%, or 99.5%.

In certain embodiments, the concentration of CoQ10 in the formulation is 1 mg/mL to 150 mg/mL. In one embodiment, the concentration of CoQ10 in the formulation is 5 mg/mL to 125 mg/mL. In one embodiment, the concentration of CoQ10 in the formulation is 10 mg/mL to 100 mg/mL. In one embodiment, the concentration of CoQ10 in the formulation is 20 mg/mL to 90 mg/mL. In one embodiment, the concentration of CoQ10 is 30 mg/mL to 80 mg/mL. In one embodiment, the concentration of CoQ10 is 30 mg/mL to 70 mg/mL. In one embodiment, the concentration of CoQ10 is 30 mg/mL to 60 mg/mL. In one embodiment, the concentration of CoQ10 is 30 mg/mL to 50 mg/mL. In one embodiment, the concentration of CoQ10 is 35 mg/mL to 45 mg/mL. It should be understood that additional ranges having any one of the foregoing values as the upper or lower limits are also intended to be part of this invention, e.g., 10 mg/mL to 50 mg/mL, or 20 mg/mL to 60 mg/mL.

In certain embodiments, the concentration of CoQ10 in the formulation is about 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90 or 95 mg/mL. In one embodiment, the concentration of CoQ10 in the formulation is about 50 mg/mL. In one embodiment, the concentration of CoQ10 in the formulation is about 60 mg/mL. In one embodiment, the concentration of CoQ10 in the formulation is about 30 mg/mL. In a preferred embodiment, the concentration of CoQ10 in the formulation is about 40 mg/mL. It should be understood that ranges having any one of these values as the upper or lower limits are also intended to be part of this invention, e.g. between 37 mg/mL and 47 mg/mL, or between 31 mg/mL and 49 mg/mL.

It is understood that formulations can similarly be prepared containing CoQ10 precursors, metabolites, and related compounds.

IV. Fatty Acid Metabolism Inhibitors

A “fatty acid metabolism inhibitor” or “inhibitor of fatty acid metabolism” as used herein, is a molecule able to inhibit, prevent or decrease fatty acid metabolism. For example, the fatty acid metabolism inhibitor may inhibit an enzyme-catalyzed reaction within a fatty acid metabolism pathway. The inhibitor may inhibit the enzyme, for example, by binding to the enzyme or otherwise interfering with operation of the enzyme and/or by reacting with a coenzyme or cofactor necessary for the enzyme to interact with a substrate.

Fatty acid metabolism pathways may include enzymes which degrade fatty acids, for example, enzymes that metabolize fatty acids within a cell for energy through the synthesis of ATP and breakdown fatty acids into simpler structures, such as CO₂, acyl groups, etc. For example, in one embodiment, the fatty acid metabolism inhibitor inhibits a beta-oxidation reaction in the fatty acid metabolism pathway. In another embodiment, the inhibitor may be an inhibitor of fatty acids as a source of energy in the mitochondria. For example, the inhibitor may inhibit the breakdown of intermediates such as butyryl CoA, glutaryl CoA, or isovaleryl CoA. Enzymes involved in the degradation of fatty acids include, but are not limited to, long-chain-fatty-acid-CoA ligase 1 (ACSL1). Fatty acid metabolism inhibitors that prevent fatty acid degradation are described, for example, in US Patent Publication Nos. 2015/0231237 and 2012/0128724, which are incorporated by reference herein in their entirety.

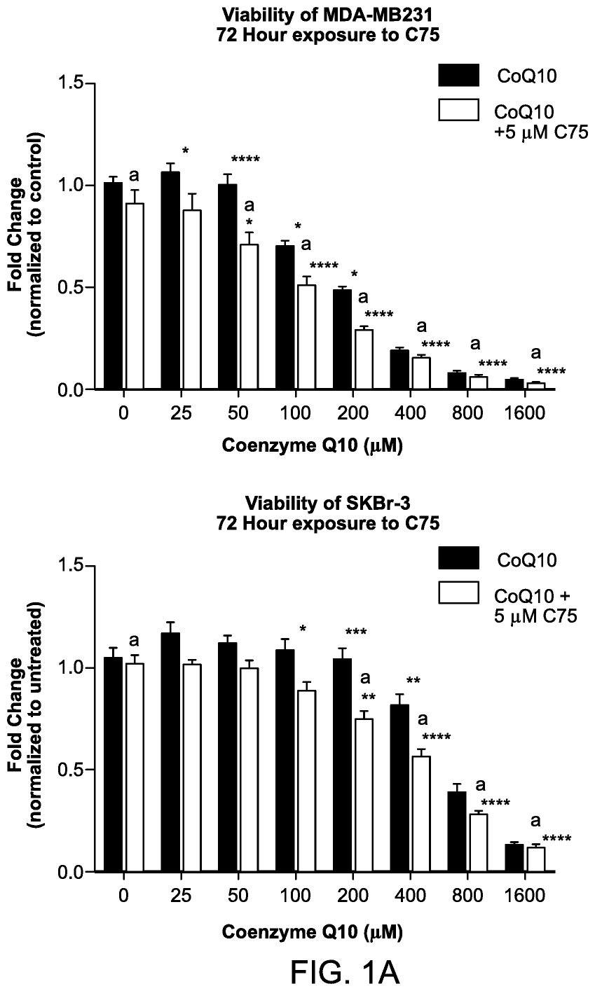

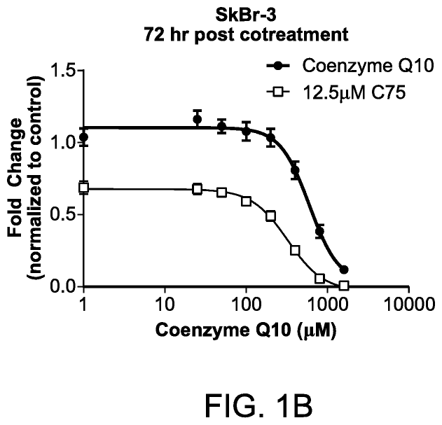

Fatty acid metabolism pathways may also include enzymes involved in the synthesis of fatty acids. Examples of enzymes involved in fatty acid synthesis include, but are not limited to, acetyl CoA carboxylase (ACC); ATP-citrate lyase (ACLY); acyl carrier protein (ACP): AMP-activated protein kinase (AMPK); carnitine palmitoyltransferase 1 (CPT1), fatty acid synthase (FASN); SREBP cleavage-activating protein (SCAP); stearoyl-CoA desaturase (SCD); sterol regulatory element binding protein (SREBP); T-cell leukemia/lymphoma 1 (TCL1); and 5 tetradecyl-oxy-Z-furoic acid (TOFA). Enzymes involved in the synthesis of fatty acids are described, for example, in Rohrig et al., 2016, Nature 16: 732-749, which is incorporated by reference herein in its entirety. In particular, FIG. 1 of Rohrig et al. shows pathways of fatty acid synthesis and uptake. Examples of fatty acid metabolism inhibitors that inhibit fatty acid synthesis are provided below in Table 4.

Fatty acid metabolism pathways also include enzymes involved in lipid storage, for example enzymes involved in fatty acid transport, and modulators of fatty acid structure (e.g. fatty acid desaturases and elongases). Accordingly, in certain embodiments, the fatty acid metabolism inhibitor is an inhibitor of a fatty acid transporter, for example, a transporter that transports fatty acids into the cell, or from the cytoplasm into the mitochondria for metabolism.

The fatty acid metabolism pathways also include several enzymatic reactions which use various enzymes such as reductases, isomerases and dehydrogenases, including but not limited to 2,4-dienoyl-CoA reductase, 2,4-dienoyl-CoA isomerase, and butyryl dehydrogenase. For example, in one embodiment, the fatty acid metabolism inhibitor is an inhibitor of 2,4-dienoyl-CoA reductase, 2,4-dienoyl-CoA isomerase, or butyryl dehydrogenase. 2,4-dienoyl-CoA reductase is an enzyme within the fatty acid metabolism pathway that catalyzes reduction reactions involved in the metabolism of polyunsaturated fatty acids. Certain fatty acids are substrates for 2,4-dienoyl-CoA reductases located within the mitochondria. In some cases, fatty acids may be transported into the mitochondria through uncoupling proteins. The uncoupling protein may, in certain instances, increase the mitochondrial metabolism to increase the availability of fatty acids within the mitochondria and/or increase the throughput of beta-oxidation within the mitochondria. The enzyme 2,4-dienoyl-CoA isomerase is an enzyme within the fatty acid metabolism pathway that catalyzes isomerization of certain fatty acids. One step in the metabolism of certain polyunsaturated fatty acids may be protective against reactive oxygen intermediates (‘ROI’). Thus, by generating substrates and antagonists for the activity of 2,4-dienyol-CoA isomerase, the metabolic production of reactive oxygen intermediates may be enhanced and/or reduced. This, in turn, may affect certain disease states, such as cancer.

In a particular embodiment, the fatty acid metabolism inhibitor is selected from the group consisting of an inhibitor of fatty acid synthase (FASN), an inhibitor of carnitine palmitoyltransferase 1 (CPT-1), an inhibitor of long-chain 3-ketoacyl-CoA thiolase, and an inhibitor of Stearoyl-CoA desaturase-1 (SCD-1). In a further particular embodiment, the fatty acid metabolism inhibitor is selected from the group consisting of C75, Etomoxir, trimetazidine and the SCD-1 inhibitor A939572. In one embodiment, the fatty acid metabolism inhibitor is C75. In one embodiment, the fatty acid metabolism inhibitor is trimetazidine. In one embodiment, the fatty acid metabolism inhibitor is Etomoxir. In one embodiment, the fatty acid metabolism inhibitor is A939572. C75 is an inhibitor of fatty acid synthase (FASN), Etomoxir is an inhibitor of carnitine palmitoyltransferase 1 (CPT-1), and trimetazidine is a β-oxidation inhibitor. C75 is described, for example, in Landree et al., 2004, J Biol Chem. 279(5):3817-27, which is incorporated by reference herein in its entirety. CPT-1 is a mitochondrial enzyme responsible for the formation of acyl carnitines by catalyzing the transfer of the acyl group of a long-chain fatty acyl-CoA from coenzyme A to 1-carnitine. The product is often palmitoylcarnitine , but other fatty acids may also be substrates. CPT-1 mediated formation of acyl carnitine allows for subsequent movement of the acyl carnitine from the cytosol into the intermembrane space of mitochondria. Trimetazidine inhibits beta-oxidation of fatty acids by blocking long-chain 3-ketoacyl-CoA thiolase, which enhances glucose oxidation. SCD-1 is an endoplasmic reticulum enzyme that catalyzes the formation of the monounsaturated fatty acids oleic acid and palmitoleic acid from stearoyl-CoA and palmitoyl-CoA. Oleic acid and palmitoleic acid are major components of membrane phospholipids, cholesterol esters and alkyl-diacylglycerol. Thus SCD-1 is a key enzyme in fatty acid metabolism. Inhibition of SCD-1 results in depletion of mono-unsaturated fatty acids and reduction of lipid storage.

Additional examples of fatty acid metabolism inhibitor suitable for use in the invention are provided below.

### Small Molecules

In certain embodiments, the fatty acid inhibitor is an oxirane carboxylic acid compound, e.g. etomoxir. In certain embodiments, the oxirane carboxylic acid compounds have the formula:

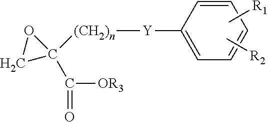

wherein: R₁ represents a hydrogen atom, a halogen atom, a 1-4C alkyl group, a 1-4C alkoxy group, a nitro group or a trifluoromethyl group; R₂ has one of the meanings of R₁; R₃ represents a hydrogen atom or a 1-4C alkyl group; Y represents the grouping —O—(CH₂)m-; m is 0 or a whole number from 1 to 4; and n is a whole number from 2 to 8 wherein the sum of m and n is a whole number from 2 to 8. In certain embodiments of the oxirane carboxylic acid compounds, R₁ is a halogen atom, R₂ is a hydrogen atom, m is 0, and n is 6, and R₃ is an ethyl group.

In a particular embodiment, the fatty acid metabolism inhibitor is etomoxir, i.e., 2-(6-(4-chlorophenoxy)-hexyl)-oxirane-2-carboxylic acid ethyl ester. Examples of other oxirane carboxylic acid compounds useful in the invention are 2-(4-(3-chlorophenoxy)-butyl)-oxirane-2-carboxylic acid ethyl ester, 2-(4-(3-trifluoromethylphenoxy)-butyl)-oxirane-2-carboxylic acid ethyl ester, 2-(5(4-chlorophenoxy)-pentyl)-oxirane-2-carboxylic acid ethyl ester, 2-(6-(3,4-dichlorophenoxy)-hexyl)-oxirane-2-carboxylic acid ethyl ester, 2-(6-(4-fluorophenoxy)-hexyl)-oxirane-2-carboxylic acid ethyl ester, and 2-(6-phenoxyhexyl)-oxirane-2-carboxylic acid ethyl ester, the corresponding oxirane carboxylic acids, and their pharmacologically acceptable salts.

The foregoing class of oxirane carboxylic acid compounds, including etomoxir, has been described by Horst Wolf and Klaus Eistetter in U.S. Pat. No. 4,946,866 for the prevention and treatment of illnesses associated with increased cholesterol and/or triglyceride concentration, and by Horst Wolf in U.S. Pat. No. 5,739,159 for treating heart insufficiency. The preparation of oxirane carboxylic acid compounds, and their use for blood glucose lowering effects as an antidiabetic agent, is described in Jew et al., U.S. Pat. No. 6,013,666. Etomoxir has been described as an inhibitor of mitochondrial carnitine palmitoyl transferase-I by Mannaerts, G. P., L. J. Debeer, J. Thomas, and P. J. De Schepper “Mitochondrial and peroxisomal fatty acid oxidation in liver homogenates and isolated hepatocytes from control and clofibrate-treated rats,” J. Biol. Chem. 254:4585-4595, 1979. United States Patent Application No. 2003/0036199 describes treating a subject having a cancer characterized by the aberrant expression of MUC 1, comprising administering to the subject etomoxir in an amount effective to reduce tumor growth.

Other, non-limiting examples of fatty acid metabolism inhibitors include fatty acid transporter inhibitors, beta-oxidation process inhibitors, reductase inhibitors, and/or isomerase inhibitors within the fatty acid metabolism pathway. Specific examples of other fatty acid metabolism inhibitors include, but are not limited to, cerulenin, 5-(tetradecyloxy)-2-furoic acid, oxfenicine, methyl palmoxirate, metoprolol, amiodarone, perhexiline, aminocarnitine, hydrazonopropionic acid, 4-bromocrotonic acid, trimetazidine, ranolazine, hypoglycin, dichloroacetate, methylene cyclopropyl acetic acid, and beta-hydroxy butyrate. As another example, the inhibitor may be a non-hydrolyzable analog of carnitine.

In certain embodiments, the fatty acid metabolism inhibitor is a carboxylic acid. In some cases, the carboxylic acid may have the structure:

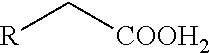

where R comprises an organic moiety, as further described below. In some cases, R may include at least two nitrogen atoms, or R may include an aromatic moiety (as further described below), such as a benzene ring, a furan, etc.

In another embodiment, the fatty acid metabolism inhibitor has the structure:

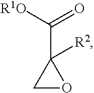

wherein each of R₁ and R₂ independently comprises an organic moiety. In some instances, either or both of R₁ and R₂ may independently be an alkyl, such as a straight-chain alkyl, for instance, methyl, ethyl, propyl, etc. In certain cases, R₂ may have at least 5 carbon atoms, at least 10 carbon atoms, or at least 15 or more carbon atoms. For example, in one embodiment, R₂ may be a tetradecyl moiety. In other cases, R₂ may include an aromatic moiety, for example, a benzene ring. In still other cases, R₂ may have the structure:

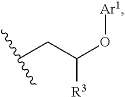

where R₃ comprises an organic moiety and Ar¹ comprises an aromatic moiety. R₃ may be an alkyl, such as a straight-chain alkyl. In some instances, Ar¹ may be a benzene ring or a derivative thereof i.e., having the structure:

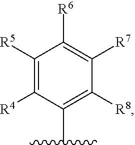

wherein each of R⁴, R⁵, R⁶, R⁷, and R⁸ is hydrogen, a halogen, an alkyl, an alkoxy, etc.

In yet another embodiment, the fatty acid metabolism inhibitor has the structure:

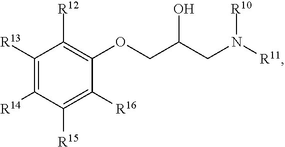

where each of R¹⁰, R¹¹, R¹², R¹³, R¹⁴ R¹⁵ and R¹⁶ independently comprises hydrogen, a halogen, or an organic moiety, such as an alkyl, an alkoxy, etc. In some cases, R¹⁰ and R¹¹ together may define an organic moiety, such as a cyclic group. For example, the fatty acid metabolism inhibitor may have the structure:

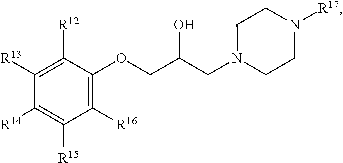

wherein R¹⁷ comprises an organic moiety, such as an alkyl, an alkoxy, an aromatic moiety, an amide, etc. An example, of R¹⁷ is:

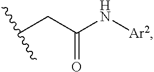

wherein Ar² comprises an aromatic moiety, such as a benzene ring or a benzene derivative, as previously described.

In other embodiments, the fatty acid metabolism inhibitor is an agent that inhibits the activity, synthesis or production of one or more enzymes within the fatty acid metabolism pathway. For example, in certain embodiments, the fatty acid metabolism inhibitor is an antisense oligonucleotide or siRNA that selectively binds to regions encoding enzymes present within the fatty acid metabolism pathway, such as 2,4-dienoyl-CoA reductase or 2,4-dienoyl-CoA isomerase. In certain embodiments, the fatty acid metabolism inhibitor is an antibody that selectively binds to an enzyme in the fatty acid metabolism pathway. Antisense and siRNA oligonucleotides and antibodies are discussed in more detail below.

Non-limiting examples of particular small molecule fatty acid metabolism inhibitors suitable for use in the methods of the invention are provided in Tables 2A-2D, Table 3 and Table 4 below.

TABLE 2A
Examples of fatty acid metabolism inhibitors under study
for cancer treatment that target lipid and cholesterol
dependent processes in cancer cells.

| Target | Drug |
|---|---|
| FASN (fatty acid synthase) | Cerulenin |
| | Orlistat |
| | C75 |
| | Triclosan |
| | EGCG |
| ACLY (ATP Citrate Lyase) | SB-204990 |
| ACC (acetyl-CoA carboxylase) | TOFA |
| LXR (liver X receptor) | T0901317 |
| | SR9243 |
| SCD1 (stearoyl-CoA desaturase-1) | A939572 |
| | CAY-10566 |
| CPT1 (carnitine palmitoyltransferase 1) | Etomoxir, |
| | Ranolazine |
| | ST1326 |
| OSC (2.3-oxydosquolene lanosteral cyclase) | RO 48-8071 |
| HMGCR (3-hydroxy-3 methyliglutaryl CoA reductase) | Statins |
| SOAT1 (sterol-O-acyl transferase 1) | Avasimibe |
| | Sandoz 5B-035 |

TABLE 2B
Examples of fatty acid metabolism inhibitors under study
for cancer treatment that modulate lipid raft components to
induce cell death signaling. Lipid raft components are combinations of
glycosphingolipids and protein receptors organized in glycolipoprotein
microdomains within plasma membranes.

| Target | Drug |
|---|---|
| Cholesterol | Methyl-β-cyclodextrine |
| LXR (liver X receptor) | T0901317 |
| | GW3965 |
| HMGCR (3-hydroxy-3 | Simvastatin |
| methyliglutaryl CoA reductase) | |
| Cell membrane | Edelfosine/Perifosine |
| Death Receptors (Fas/TRAIL | Avicin D |
| (tumor necrosis factor-related | Resveratrol |
| apoptosis-inducing ligand) | |

TABLE 2C
Examples of fatty acid metabolism inhibitors under study for cancer
treatment that disrupt lipid homeostasis to induce ER stress and apoptosis.

| Target | Drug |
|---|---|
| Site-1 and Site-2 proteases | Nelfinavir |
| FASN (fatty acid synthase) | Orlistate |
| | C75 |
| SOAT1 (sterol-O-acyl transferase 1) | Mitotane |
| | Sandoz 5-8-035 |
| SCD1 (stearoyl-CoA desaturase-1) | A939572 |
| Ceramide accumulation | Cannabinoids |

TABLE 2D
Examples of fatty acid metabolism inhibitors under study for cancer
treatment that target lipid mediators of tumor-stroma dialog.

| Target | Drug |
|---|---|
| Cox-2 (cyclo-oxygenase-2) | Celecoxib |
| | Cg10006-49 |
| PGE2 (prostaglandin E2) receptors | Sc-51322 |
| | AH6809 |
| | AH23848B |
| | ONO-AE3-208 |
| | ONO-8711 |
| S1P (sphingosine-1-phosphate) | Sphingomab |
| SPHK1 (sphingosine kinase 1) | SK1-L |
| S1PR1 (S1P receptor 1) | FTY720 |

TABLE 3
Examples of fatty acid metabolism inhibitors that inhibit lipid
enzymes and reduce fatty acid availability.

| Enzyme | Inhibitor | Comments |
|---|---|---|
| ACC | Soraphen-A | |
| | TOFA | |
| | (5-(TETRADECYLOXY)- | |
| | 2-furoic acid) | |
| | A-7-69662 | |
| | Metformin | Indirect, activates |
| | | AMPK, FDA approved |
| | ALCAR | Indirect, activates AMPK |
| ACLY | SIB-204990 | |
| | LY294002 | |
| ACS | Triacscin C | |
| | Thiazolidinediones | ACSL4 specific, also activates |
| | (TZDs) | PPAR [symbol] , FDA approved |
| AGPAT | CT-32501 | AGPAT2 specific |
| CKα | TCD-717 | Currently in phase I trials |
| CIC | Benzene-tricarboxylate | |
| | analog (BTA) | |
| CPT1 | Etomoxir | |
| | Ranolazine | FDA approved |
| FASN | Cerulenin and its derivative | |
| | C75 | |
| | Orlistate | FDA approved |
| | Flavonoids | Naturally occurring |
| | Epigalocatechin-3-gallate | Found in green tea |
| | (EGCG) | |
| MAGL | JZL184 | |
| SCD | BZ36 | |
| | A939572 | |
| SREBP | Fatostatin | Inhibits processing of |
| | | SREBP-1 and SREBP-2 |
| MAGL | FGH10019 | Inhibits processing of |
| | | SREBP-1 and SREBP-2 |

ACC, acetyl CoA carboxylase; ACLY, ATP-citrate lyase; ACS, long fatty acyl CoA synthetase; AGPAT, 1-acylglycerol-3-phosphate-O-acyltransferase ; CKα, choline kinase alpha; CPT1, carnitine palmitoyltransferase 1; FASN, fatty acid synthase; MAGL, Monoacylglycerol lipase; SREBP cleavage-activating protein: SCD, stearoyl-CoA desaturase: SREBP, sterol regulatory element binding protein.

TABLE 4
Fatty acid metabolism inhibitors that inhibit fatty acid synthesis.

| Inhibitor | Activity |
|---|---|
| TVB-2640 | NA |
| TVB-3166 | Inhibition of the β-ketoacyl-reductase activity |
| GSK2194069 | Inhibition of the β-ketoacyl-reductase activity |
| C93 | Inhibition of the β-ketoacyl-reductase activity |
| FAS31 | Not known |
| C247 | Not known |
| C75 | Inhibition of the β-ketoacyl-reductase activity |
| Cerulenin | Inhibition of the β-ketoacyl-synthase activity |
| Orlistat | Inhibition of thioesterase domain |
| Triclosan | Inhibition of enoyl-ACP reductase domain |
| TOFA | Inhibition of ACCs |
| Soraphen A | Inhibition of ACCs |
| SB-204990 | Inhibition of ACLY |
| Triacscin C | Inhibition of acyl-CoA synthatases |
| Etomoxir | Inhibition of CPT1 |
| Perhexiline | Inhibition of CPT1 |
| BZ36 | Inhibition of SCD |
| A939572 | Inhibition of SCD |
| Fatostatin | Inhibition of SREBP-SCAP interaction |
| Betulin | Inhibition of SREBP-SCAP interaction |

ACC, acetyl CoA carboxylase; ACLY, ATP-citrate lyase; ACP, acyl carrier protein: AMPK, AMP-activated protein kinase: CPT1, carnitine palmitoyltransferase 1: FASN, fatty acid synthase: NA, not available: SCAP, SREBP cleavage-activating protein: SCD, stearoyl-CoA desaturase: SREBP, sterol regulatory element binding protein: TCL1, T-cell leukemia/lymphoma 1: TOFA, 5 tetradecyl-oxy-Z-furoic acid.
(See Rohrig et al., 2016, cited above, page 743, Table 1.)

### Single Stranded Nucleic Acids

In certain embodiments, the fatty acid metabolism inhibitor is a single stranded nucleic acid, for example, an antisense nucleic acid that targets a nucleic acid sequence that encodes a protein involved in fatty acid metabolism. Antisense nucleic acids are single stranded nucleic acids, typically about 16 to 30 nucleotides in length, and are complementary to a target nucleic acid sequence in the target cell, either in culture or in an organism, e.g. a nucleic acid sequence that encodes a protein involved in fatty acid metabolism.

In some embodiments, the inhibitor is a single-stranded antisense RNA molecule, a single-stranded antisense DNA molecule, or a single-stranded antisense polynucleotide comprising both DNA and RNA. In a particular embodiment, the antisense molecule is an antisense oligonucleotide (ASO) comprising both DNA and RNA. An antisense molecule is complementary to a sequence within the target mRNA. Antisense molecules can inhibit translation in a stoichiometric manner by base pairing to the mRNA and physically obstructing the translation machinery, see Dias, N. et al., (2002) Mol Cancer Ther 1:347-355. The antisense molecule may have about 15-30 nucleotides that are complementary to the target mRNA. For example, the antisense molecule may have a sequence of at least 15, 16, 17, 18, 19, 20, 21, 22, 23, 24 or 25 or more contiguous nucleotides that are complementary to the target mRNA.

In some embodiments, the ASO comprises at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. Any of these values may be used to define a range for the number of nucleotides in the ASO. For example, the ASO may comprise at least 8-50, 15-30, or 20-25 nucleotides. In some embodiments, the ASO consists of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. Any of these values may be used to define a range for the number of nucleotides in the ASO. For example, the ASO may consist of 8-50, 15-30, or 20-25 nucleotides.

Patents directed to antisense nucleic acids, chemical modifications, and therapeutic uses are provided, for example, in U.S. Pat. No. 5,898,031 related to chemically modified RNA-containing therapeutic compounds, and U.S. Pat. No. 6,107,094 related methods of using these compounds as therapeutic agent. U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality RNA nucleosides and at least one chemical modification. The entire contents of each of the patents listed in this paragraph are incorporated herein by reference. In one aspect of the invention, the agent is a single-stranded antisense nucleic acid molecule (ASO). Antisense oligonucleotides (ASOs) are synthetic molecules approximately 18-21 nucleotides in length and complementary to the mRNA sequence of the target gene. ASOs bind cognate mRNA sequences through sequence-specific hybridization resulting in cleavage or disablement of the mRNA and inhibition of the expression of the target gene (reviewed in Mansoor M and Melemdez M. Gene Regulation and Systems Biology 2008:2 275-295).

### Double Stranded Nucleic Acids

In certain embodiments, the fatty acid metabolism inhibitor is a double stranded nucleic acid therapeutic. In particular embodiments, the double stranded nucleic acid binds to a nucleic acid encoding a protein involved in fatty acid metabolism. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi agent may comprise one or more nucleotide overhangs. The term “siRNA” is also used herein to refer to an RNAi agent as described above.

In many embodiments, the duplex region is 15-30 nucleotide pairs in length. In some embodiments, the duplex region is 17-23 nucleotide pairs in length, 17-25 nucleotide pairs in length, 23-27 nucleotide pairs in length, 19-21 nucleotide pairs in length, or 21-23 nucleotide pairs in length. In certain embodiments, each strand has 15-30 nucleotides.

The RNAi agents that are used in the methods of the invention include agents with chemical modifications as disclosed, for example, in U.S. Provisional Application No. 61/561,710, filed on Nov. 18, 2011, International Application No. PCT/US2011/051597, filed on Sep. 15, 2010, and PCT Publication WO 2009/073809, the entire contents of each of which are incorporated herein by reference. The term “antisense strand” refers to the strand of a double stranded RNAi agent which includes a region that is substantially complementary to a target sequence (e.g., a human TTR mRNA). As used herein, the term “region complementary to part of an mRNA encoding transthyretin” refers to a region on the antisense strand that is substantially complementary to part of a TTR mRNA sequence. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

### Antibodies

In certain embodiments, the fatty acid metabolism inhibitor is an antibody, including a polyclonal or monoclonal antibody, or an antigen-binding portion thereof, that binds to a molecule, e.g., a protein, involved in fatty acid metabolism. The term “monoclonal antibody” or “monoclonal antibody composition”, as used herein, refers to a population of antibody molecules that contain only one species of an antigen binding site capable of immunoreacting with a particular epitope. Preferred antibodies for use in the invention include monoclonal antibodies that bind to proteins involved in fatty acid metabolism. Antibodies can be obtained from commercial sources or produced using known methods.

Polyclonal antibodies can be prepared by immunizing a suitable subject with a protein of the invention as an immunogen. The antibody titer in the immunized subject can be monitored over time by standard techniques, such as with an enzyme linked immunosorbent assay (ELISA) using immobilized polypeptide. At an appropriate time after immunization, e.g., when the specific antibody titers are highest, antibody-producing cells can be obtained from the subject and used to prepare monoclonal antibodies (mAb) by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a protein of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Recombinant antibodies that specifically bind a protein of interest can also be used in the methods of the invention. In preferred embodiments, the recombinant antibodies specifically binds a protein of interest or fragment thereof. Recombinant antibodies include, but are not limited to, chimeric and humanized monoclonal antibodies, comprising both human and non-human portions, single-chain antibodies and multi-specific antibodies. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Single-chain antibodies have an antigen binding site and consist of a single polypeptide. They can be produced by techniques known in the art, for example using methods described in Ladner et. al U.S. Pat. No. 4,946,778 (which is incorporated herein by reference in its entirety); Bird et al., (1988) Science 242:423-426; Whitlow et al., (1991) Methods in Enzymology 2:1-9; Whitlow et al., (1991) Methods in Enzymology 2:97-105; and Huston et al., (1991) Methods in Enzymology Molecular Design and Modeling: Concepts and Applications 203:46-88. Multi-specific antibodies are antibody molecules having at least two antigen-binding sites that specifically bind different antigens. Such molecules can be produced by techniques known in the art, for example using methods described in Segal, U.S. Pat. No. 4,676,980 (the disclosure of which is incorporated herein by reference in its entirety); Holliger et al., (1993) Proc. Natl. Acad. Sci. USA 90:6444-6448; Whitlow et al., (1994) Protein Eng. 7:1017-1026 and U.S. Pat. No. 6,121,424.

Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

More particularly, humanized antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

The antibodies of the invention can be isolated after production (e.g., from the blood or serum of the subject) or synthesis and further purified by well-known techniques. For example, IgG antibodies can be purified using protein A chromatography. Antibodies specific for a protein of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein of the invention.

Dominant Negative Polypeptides

In still another embodiment, the fatty acid metabolism inhibitor includes a dominant negative polypeptide which reduces the function of enzymes present within the fatty acid metabolism pathway. One of ordinary skill in the art can assess the potential for a dominant negative variant of a protein or enzyme, and use standard mutagenesis techniques to create one or more dominant negative variant polypeptides. For example, one of ordinary skill in the art can modify the sequence of an enzyme coding region by site-specific mutagenesis, scanning mutagenesis, partial gene deletion or truncation, and the like. See, e.g., U.S. Pat. No. 5,580,723 and Sambrook, et al., Molecular Cloning: A Laboratory Manual, Second Edition, Cold Spring Harbor Laboratory Press, 1989. One of ordinary skill in the art then can test the population of mutagenized polypeptides for diminution in a selected activity of the protein or enzyme and/or for retention of such activity of the protein or enzyme. Other similar methods for creating and testing dominant negative variants of a protein will be apparent to one of ordinary skill in the art.

### V. Combination Therapies

Provided herein are methods of treating oncological disorders in a subject by co-administering CoQ10 and at least one fatty acid metabolism inhibitor to a subject in need thereof. As used herein, the term “co-administering” refers to administration of CoQ10 prior to, concurrently or substantially concurrently with, subsequently to, or intermittently with the administration of the fatty acid metabolism inhibitor. In certain embodiments, CoQ10 is administered prior to and concurrently with the fatty acid metabolism inhibitor. In certain embodiments, CoQ10 is administered prior to but not concurrently with the fatty acid metabolism inhibitor, i.e., CoQ10 administration is discontinued prior to initiation of treatment with or administration of a fatty acid metabolism inhibitor.

In one embodiment, an intravenous (IV) CoQ10 formulation can be used in combination therapy with at least one fatty acid metabolism inhibitor according to the methods of the invention. In one embodiment, a topical CoQ10 formulation can be used in combination therapy with at least one fatty acid metabolism inhibitor according to the methods of the invention. In one embodiment, an inhalable CoQ10 formulation can be used in combination therapy with at least one fatty acid metabolism inhibitor according to the methods of the invention. CoQ10 and/or pharmaceutical formulations thereof and the fatty acid metabolism inhibitor can act additively or, more preferably, synergistically. In one embodiment, CoQ10 and/or a formulation thereof is administered concurrently with the administration of the fatty acid metabolism inhibitor. In another embodiment, CoQ10 and/or pharmaceutical formulation thereof is administered prior to or subsequent to administration of the fatty acid metabolism inhibitor. In one embodiment, the CoQ10 and fatty acid metabolism inhibitor act synergistically. In some embodiments the synergistic results are in the treatment of the oncological disorder. In other embodiments the synergistic results are in modulation of the toxicity associated with the fatty acid metabolism inhibitor. In one embodiment, the CoQ10 and the fatty acid inhibitor act additively. In one embodiment, the CoQ10 sensitizes the oncological disorder, cancer or cancer cells to treatment with the fatty acid metabolism inhibitor. In one embodiment, pre-treatment with CoQ10 prior to treatment with the fatty acid metabolism inhibitor sensitizes the oncological disorder, cancer or cancer cells to treatment with the fatty acid metabolism inhibitor. In one embodiment, pre-treatment with CoQ10 and discontinuation of said treatment prior to treatment with the fatty acid metabolism inhibitor sensitizes the oncological disorder, cancer or cancer cells to treatment with the fatty acid metabolism inhibitor.

In some embodiments, the CoQ10 is in the form of an intravenous CoQ10 formulation, an inhalation CoQ10 formulation, or a topical CoQ10 formulation. Intravenous CoQ10 formulations are disclosed in WO2011/112900, filed on Mar. 11, 2011. The disclosure of WO2011/112900 is incorporated herein in its entirety. Topical CoQ10 formulations are disclosed in US Patent Application Publication No. US2011/0027247, filed on May 11, 2010. The disclosure of US2011/0027247 is incorporated herein in its entirety. Inhalation CoQ10 formulations are disclosed in US Patent Publication Nos. 20120321698, filed on Jun. 18, 2012 and 20110142914 filed Dec. 5, 2008. The CoQ10 and the fatty acid metabolism inhibitor need not be delivered by the same route of administration. In certain embodiments, the CoQ10 is administered orally. In certain embodiments, the CoQ10 is not administered orally.

In some embodiments, methods are provided for the treatment of oncological disorders by co-administering intravenous CoQ10 formulations with a fatty acid metabolism inhibitor. In certain embodiments, the fatty acid metabolism inhibitor is selected from the group consisting of C75, Etomoxir, trimetazidine, inhibitors of fatty acid synthase (FASN), carnitine palmitoyltransferase 1 (CPT-1) and a β-oxidation inhibitor.

In some embodiments, the Coenzyme Q10 and at least one fatty acid metabolism inhibitor are administered in combination with at least one additional anticancer agent, such as a chemotherapeutic agent. In certain embodiments, the anticancer, e.g., chemotherapeutic agent is an antimetabolites or an anthracycline. Chemotherapeutic agents generally belong to various classes including, for example: 1. Topoisomerase II inhibitors (cytotoxic antibiotics), such as the anthracyclines/anthracenediones, e.g., doxorubicin, epirubicin, idarubicin and nemorubicin, the anthraquinones, e.g., mitoxantrone and losoxantrone, and the podophillotoxines, e.g., etoposide and teniposide; 2. Agents that affect microtubule formation (mitotic inhibitors), such as plant alkaloids (e.g., a compound belonging to a family of alkaline, nitrogen-containing molecules derived from plants that are biologically active and cytotoxic), e.g., taxanes, e.g., paclitaxel and docetaxel, and the vinka alkaloids, e.g., vinblastine, vincristine, and vinorelbine, and derivatives of podophyllotoxin; 3. Alkylating agents, such as nitrogen mustards, ethyleneimine compounds, alkyl sulphonates and other compounds with an alkylating action such as nitrosoureas, dacarbazine, cyclophosphamide, ifosfamide and melphalan; 4. Antimetabolites (nucleoside inhibitors), for example, folates, e.g., folic acid, fiuropyrimidines, purine or pyrimidine analogues such as 5-fluorouracil, capecitabine, gemcitabine, methotrexate and edatrexate; 5. Topoisomerase I inhibitors, such as topotecan, irinotecan, and 9-nitrocamptothecin, and camptothecin derivatives; and 6. Platinum compounds/complexes, such as cisplatin, oxaliplatin, and carboplatin.

Exemplary anticancer agents for use in the methods of the invention include, but are not limited to, amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-I1, IO-hydroxy-7-ethyl-camptothecin (SN38), dacarbazine, S-I capecitabine, ftorafur, 5′deoxyflurouridine, UFT, eniluracil, deoxycytidine, 5-azacytosine, 5-azadeoxycytosine, allopurinol, 2-chloro adenosine, trimetrexate, aminopterin, methylene-10-deazaaminopterin (MDAM), oxaplatin, picoplatin, tetraplatin, satraplatin, platinum-DACH, ormaplatin, CI-973, JM-216, and analogs thereof, epirubicin, etoposide phosphate, 9-aminocamptothecin, 10, 11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, Capecitabine, Pentostatin, Trimetrexate, Cladribine, floxuridine, fludarabine, hydroxyurea, ifosfamide, idarubicin, mesna, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, cisplatin, doxorubicin, paclitaxel (taxol), bleomycin, mTor, epidermal growth factor receptor (EGFR), and fibroblast growth factors (FGF) and combinations thereof which are readily apparent to one of skill in the art based on the appropriate standard of care for a particular tumor or cancer.

In certain embodiments, an additional anticancer agent for use in the combination therapies of the invention is a biologic agent. Biologic agents (also called biologics) are the products of a biological system, e.g., an organism, cell, or recombinant system. Examples of such biologic agents include nucleic acid molecules (e.g., antisense nucleic acid molecules), interferons, interleukins, colony-stimulating factors, antibodies, e.g., monoclonal antibodies, anti-angiogenesis agents, and cytokines. Exemplary biologic agents are discussed in more detail below and generally belong to various classes including, for example: 1. Hormones, hormonal analogues, and hormonal complexes, e.g., estrogens and estrogen analogs, progesterone, progesterone analogs and progestins, androgens, adrenocorticosteroids, antiestrogens, antiandrogens, antitestosterones, adrenal steroid inhibitors, and anti-leuteinizing hormones; and 2. Enzymes, proteins, peptides, polyclonal and/or monoclonal antibodies, such as interleukins, interferons, colony stimulating factor, etc.

In one embodiment, the biologic is an interferon. Interferons (IFN) are a type biologic agent that naturally occurs in the body. Interferons are also produced in the laboratory and given to cancer patients in biological therapy. They have been shown to improve the way a cancer patient's immune system acts against cancer cells.

Interferons may work directly on cancer cells to slow their growth, or they may cause cancer cells to change into cells with more normal behavior. Some interferons may also stimulate natural killer cells (NK) cells, T cells, and macrophages which are types of white blood cells in the bloodstream that help to fight cancer cells.

In one embodiment, the biologic is an interleukin. Interleukins (IL) stimulate the growth and activity of many immune cells. They are proteins (cytokines and chemokines) that occur naturally in the body, but can also be made in the laboratory. Some interleukins stimulate the growth and activity of immune cells, such as lymphocytes, which work to destroy cancer cells.

In another embodiment, the biologic is a colony-stimulating factor. Colony-stimulating factors (CSFs) are proteins given to patients to encourage stem cells within the bone marrow to produce more blood cells. The body constantly needs new white blood cells, red blood cells, and platelets, especially when cancer is present. CSFs are given, along with chemotherapy, to help boost the immune system. When cancer patients receive chemotherapy, the bone marrow's ability to produce new blood cells is suppressed, making patients more prone to developing infections. Parts of the immune system cannot function without blood cells, thus colony-stimulating factors encourage the bone marrow stem cells to produce white blood cells, platelets, and red blood cells. With proper cell production, other cancer treatments can continue enabling patients to safely receive higher doses of chemotherapy.

In another embodiment, the biologic is an antibody. Antibodies, e.g., monoclonal antibodies, are agents, produced in the laboratory, that bind to cancer cells.

Monoclonal antibody agents do not destroy healthy cells. Monoclonal antibodies achieve their therapeutic effect through various mechanisms. They can have direct effects in producing apoptosis or programmed cell death. They can block growth factor receptors, effectively arresting proliferation of tumor cells. In cells that express monoclonal antibodies, they can bring about anti-idiotype antibody formation.

Examples of antibodies which may be used in the combination treatment of the invention include anti-CD20 antibodies, such as, but not limited to, cetuximab, Tositumomab, rituximab, and Ibritumomab. Anti-HER2 antibodies may also be used in combination with coenzyme Q10 for the treatment of cancer. In one embodiment, the anti-HER2 antibody is Trastuzumab (Herceptin). Other examples of antibodies which may be used in combination with coenzyme Q10 for the treatment of cancer include anti-CD52 antibodies (e.g., Alemtuzumab), anti-CD-22 antibodies (e.g., Epratuzumab), and anti-CD33 antibodies (e.g., Gemtuzumab ozogamicin). Anti-VEGF antibodies may also be used in combination with coenzyme Q10 for the treatment of cancer. In one embodiment, the anti-VEGF antibody is bevacizumab. In other embodiments, the biologic agent is an antibody which is an anti-EGFR antibody e.g., cetuximab. Another example is the anti-glycoprotein 17-1A antibody edrecolomab. Numerous other anti-tumor antibodies are known in the art and would be understood by the skilled artisan to be encompassed by the present invention.

In another embodiment, the biologic is a cytokine. Cytokine therapy uses proteins (cytokines) to help a subject's immune system recognize and destroy those cells that are cancerous. Cytokines are produced naturally in the body by the immune system, but can also be produced in the laboratory. This therapy is used with advanced melanoma and with adjuvant therapy (therapy given after or in addition to the primary cancer treatment). Cytokine therapy reaches all parts of the body to kill cancer cells and prevent tumors from growing.

In another embodiment, the biologic is a fusion protein. For example, recombinant human Apo2L/TRAIL (GENETECH) may be used in a combination therapy. Apo2/TRAIL is the first dual pro-apoptotic receptor agonist designed to activate both pro-apoptotic receptors DR4 and DRS, which are involved in the regulation of apoptosis (programmed cell death).

In one embodiment, the biologic is a therapeutic nucleic acid molecule. Nucleic acid therapeutics are well known in the art. Nucleic acid therapeutics include both single stranded and double stranded (i.e., nucleic acid therapeutics having a complementary region of at least 15 nucleotides in length) nucleic acids that are complementary to a target sequence in a cell. Therapeutic nucleic acids can be directed against essentially any target nucleic acid sequence in a cell. In certain embodiments, the nucleic acid therapeutic is targeted against a nucleic acid sequence encoding a stimulator of angiogenesis, e.g., VEGF, FGF, or of tumor growth, e.g., EGFR.

Antisense nucleic acid therapeutic agents are single stranded nucleic acid therapeutics, typically about 16 to 30 nucleotides in length, and are complementary to a target nucleic acid sequence in the target cell, either in culture or in an organism.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. Patents directed to antisense nucleic acids, chemical modifications, and therapeutic uses are provided, for example, in U.S. Pat. No. 5,898,031 related to chemically modified RNA-containing therapeutic compounds, and U.S. Pat. No. 6,107,094 related methods of using these compounds as therapeutic agent. U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality RNA nucleosides and at least one chemical modification. The entire contents of each of the patents listed in this paragraph are incorporated herein by reference.

Nucleic acid therapeutic agents for use in the methods of the invention also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in U.S. Provisional Application No. 61/561,710, filed on Nov. 18, 2011, International Application No. PCT/US2011/051597, filed on Sep. 15, 2010, and PCT Publication WO 2009/073809, the entire contents of each of which are incorporated herein by reference.

Additional exemplary biologic agents for use in the methods of the invention include, but are not limited to, gefitinib (Iressa), anastrazole, diethylstilbesterol, estradiol, premarin, raloxifene, progesterone, norethynodrel, esthisterone, dimesthisterone, megestrol acetate, medroxyprogesterone acetate, hydroxyprogesterone caproate, norethisterone, methyltestosterone, testosterone, dexamthasone, prednisone, Cortisol, solumedrol, tamoxifen, fulvestrant, toremifene, aminoglutethimide, testolactone, droloxifene, anastrozole, bicalutamide, flutamide, nilutamide, goserelin, flutamide, leuprolide, triptorelin, aminoglutethimide, mitotane, goserelin, cetuximab, erlotinib, imatinib, Tositumomab, Alemtuzumab, Trastuzumab, Gemtuzumab, Rituximab, Ibritumomab tiuxetan, Bevacizumab, Denileukin diftitox, Daclizumab, interferon alpha, interferon beta, anti-4-lBB, anti-4-blBBL, anti-CD40, anti-CD 154, anti- OX40, anti-OX40L, anti-CD28, anti-CD80, anti-CD86, anti-CD70, anti-CD27, anti- HVEM, anti-LIGHT, anti-GITR, anti-GITRL, anti-CTLA-4, soluble OX40L, soluble 4-IBBL, soluble CD154, soluble GITRL, soluble LIGHT, soluble CD70, soluble CD80, soluble CD86, soluble CTLA4-Ig, GVAX®, and combinations thereof which are readily apparent to one of skill in the art based on the appropriate standard of care for a particular tumor or cancer. The soluble forms of agents may be made as, for example fusion proteins, by operatively linking the agent with, for example, Ig-Fc region.

It should be noted that more than one additional anticancer agent (e.g., chemotherapeutic agent), e.g., 2, 3, 4, 5, or more, may be administered in combination with the coenzyme Q10 and fatty acid metabolism inhibitors provided herein. For example, in one embodiment, two additional anticancer agents (e.g., chemotherapeutic agents) may be administered in combination with coenzyme Q10 and the at least one fatty acid metabolism inhibitor. In one embodiment, three additional anticancer agents (e.g., chemotherapeutic agents) may be administered in combination with coenzyme Q10 and the at least one fatty acid metabolism inhibitor. Appropriate doses and routes of administration of the anticancer agents (e.g., chemotherapeutic agents) provided herein are known in the art.

In certain embodiments, the methods of the invention comprise treatment of cancer by continuous infusion of coenzyme Q10 provided and combination therapies with additional anticancer agents or interventions (e.g., radiation, surgery, bone marrow transplant). In certain embodiments, “combination therapy” includes a treatment with coenzyme Q10 to decrease tumor burden and/or improve clinical response. Administration of coenzyme Q10 with palliative treatments or treatments to mitigate drug side effects (e.g., to decrease nausea, pain, anxiety, or inflammation, to normalize clotting) is not considered to be a combination treatment of the cancer.

In certain embodiments, treatment of subjects with leukemia, particularly ALL or AML, administration (e.g., intravenous, e.g., continuous infusion) of coenzyme Q10 is combined with one, or preferably both, of the following treatments.

1. Fludarabine, preferably at a dose of 15 mg/m² administered intravenously over 15-30 minutes ±15 minutes, every 12 hours for 5 days (or for 4 days in patients over 65 years of age or with ECOG Performance Status of 3).

2. Cytarabine, preferably administered at 0.5 g/m² in 250 ml of normal saline administered intravenously over 2 hours ±20 minutes every 12 hours ±2 hours for 5 days (or for 4 days in patients over 65 years of age or with ECOG Performance Status of 3).

In certain embodiments, 1, 2, 3, 4, or 5 cycles of the combination therapy are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

In certain embodiments, treatment of subjects with solid tumors by continuous infusion of coenzyme Q10 is combined with one or more of the following treatments.

1. Gemcitabine, preferably by intravenous administration at a weekly dose starting at 600 mg/m², with the dose being adjusted based on the tolerance of the subject to the drug.

2. 5-Fluorouracil (5-FU), preferably by intravenous administration at a weekly starting dose of 350 mg/m², with the dose being adjusted based on the tolerance of the subject to the drug, in combination with leucovorin at 100 mg/m².

3. Docetaxel, preferably by intravenous administration once weekly at a starting dose of 20 mg/m², with the dose being adjusted based on the tolerance of the subject to the drug.

In certain embodiments, 1, 2, 3, 4, or 5 cycles of the combination therapy are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

In other embodiments, the fatty acid metabolism inhibitor is administered at a dosage that is lower than the standard dosages of the fatty acid metabolism inhibitor used to treat the oncological disorder under the standard of care for treatment for a particular oncological disorder. Standard dosages of fatty acid metabolism inhibitors are known to a person skilled in the art and may be obtained, for example, from the product insert provided by the manufacturer of the fatty acid metabolism inhibitor. In certain embodiments, the dosage administered of the fatty acid metabolism inhibitor is 5%, 10%, 20%, 30%, 40%, 50%, 60%, 70%, 80%, or 90% lower than the standard dosage of the fatty acid metabolism inhibitor for a particular oncological disorder. In certain embodiments, the dosage administered of the fatty acid metabolism inhibitor is 95%, 90%, 85%, 80%, 75%, 70%, 65%, 60%, 55%, 50%, 45%, 40%, 35%, 30%, 25%, 20%, 15%, 10% or 5% of the standard dosage of the fatty acid metabolism inhibitor for a particular oncological disorder.

In other embodiments, the Coenzyme Q10 is administered at a dosage that is lower than the standard dosages of Coenzyme Q10 used to treat the oncological disorder under the standard of care for treatment for a particular oncological disorder. Standard dosages of Coenzyme Q10 are known to a person skilled in the art and are described herein. In certain embodiments, the dosage administered of Coenzyme Q10 is 5%, 10%, 20%, 30%, 40%, 50%, 60%, 70%, 80%, or 90% lower than the standard dosage of Coenzyme Q10 for a particular oncological disorder. In certain embodiments, the dosage administered of Coenzyme Q10 is 95%, 90%, 85%, 80%, 75%, 70%, 65%, 60%, 55%, 50%, 45%, 40%, 35%, 30%, 25%, 20%, 15%, 10% or 5% of the standard dosage of Coenzyme Q10 for a particular oncological disorder.

### VI. Treatment of Oncological Disorders

The combination therapies of the present invention, e.g., a CoQ10 compound and a fatty acid metabolism inhibitor, may be utilized for the treatment of oncological disorders. Accordingly, the present invention provides methods of treating or preventing an oncological disorder in a subject, comprising administering the formulations of the invention to the subject in an amount sufficient to treat or prevent the oncological disorder, thereby treating or preventing the oncological disorder. The formulations of the invention may also be utilized for inhibiting tumor cell growth. Accordingly, the invention further provides methods of inhibiting tumor cell growth in a subject, comprising intravenously administering the formulations of the invention to the subject, such that tumor cell growth is inhibited. In certain embodiments, treating cancer comprises extending survival or extending time to tumor progression as compared to control, e.g., a population control. In certain embodiments, the subject is a human subject. In preferred embodiments, the subject is identified as having a tumor prior to administration of the first dose of CoQ10. In certain embodiments, the subject has a tumor at the time of the first administration of CoQ10.

Such combination therapies include, for example, CoQ10 formulations that are co-administered with the fatty acid metabolism inhibitors described or incorporated herein. In certain embodiments, the method of treating an oncological disorder in a subject comprises: (a) administering coenzyme Q10 (CoQ10) to the subject; (b) discontinuing treatment with CoQ10; and (c) administering at least one fatty acid metabolism inhibitor to the subject after administration of CoQ10 has been discontinued, wherein the oncological disorder is treated.

In other embodiments, the method of treating an oncological disorder in a subject comprises: (a) administering coenzyme Q10 (CoQ10) to the subject; (b) administering at least one fatty acid metabolism inhibitor to the subject after administration of the CoQ10 is initiated; and (c) continuing treatment with CoQ10 after administration of the at least one fatty acid metabolism inhibitor is initiated, wherein the oncological disorder is treated.

In other embodiments, the method of treating an oncological disorder in a subject comprises: pre-treating a subject having an oncological disorder with Coenzyme Q10 (CoQ10) for a sufficient time prior to initiation of administration of one or more fatty acid metabolism inhibitors, such that a response of the oncological disorder is improved relative to treatment with the CoQ10 alone and/or the fatty acid metabolism inhibitor alone.

In yet other embodiments, the method of treating an oncological disorder in a subject comprises: (a) administering coenzyme Q10 (CoQ10) to the subject; and (b) administering at least one fatty acid metabolism inhibitor to the subject at a dosage that is lower than standard dosages of the fatty acid metabolism inhibitor used to treat the oncological disorder, such that the oncological disorder is treated.

In still other embodiments, the method of treating an oncological disorder in a subject comprises: (a) administering at least one fatty acid metabolism inhibitor to the subject; and (b) administering coenzyme Q10 (CoQ10) to the subject at a dosage that is lower than standard dosages of coenzyme Q10 (CoQ10) used to treat the oncological disorder, such that the oncological disorder is treated.

The CoQ10 formulations may be inhalation formulations, intravenous formulations, oral formulations or topical formulations. In certain embodiments, the CoQ10 formulation is not an oral formulation. For example, the intravenous formulations may include CoQ10 or its metabolites, in a pharmaceutically acceptable carrier. In some embodiments, such a formulation may include from about 0.001% to about 20% (w/w) of CoQ10, more preferably between about 0.01% and about 15% and even more preferably between about 0.1% to about 10% (w/w) of CoQ10, more preferably about 3% to about 5% (w/w) of CoQ10. In one embodiment a formulation includes about 4% (w/w) of CoQ10. In one embodiment a formulation includes about 8% (w/w) of CoQ10. In various embodiments, the formulation includes about 0.5%, 1%, 2%, 3%, 4%, 5%, 6%, 7%, 8%, 9%, 10%, 11%, 12%, 13%, 14%, 15%, 16%, 17%, 18%, 19% or 20% (w/w) of CoQ10. As also noted herein, compositions of the present disclosure may be in a liquid form, capable of introduction into a subject by any means or route of administration within the purview of those skilled in the art. For example, compositions may be administered by routes of administration including, but not limited to, intravenous, intratumoral, intraperitoneal, combinations thereof, and the like.

In certain embodiments, a loading dose of CoQ10 is administered prior to administration of the fatty acid metabolism inhibitor. In certain embodiments, CoQ10 is administered to achieve a steady state level of CoQ10 prior to administration of the fatty acid metabolism inhibitor. Where the combination therapy includes intravenous CoQ10 formulations, the subject is intravenously administered the CoQ10 such that oncological disorders are treated or prevented. In one embodiment, the subject is intravenously administered the CoQ10 such that response to the fatty acid metabolism inhibitor is improved, e.g., relative to treatment with the fatty acid metabolism inhibitor alone.

The subject is administered a dose of CoQ10 in the range of about 0.5 mg/kg to about 10,000 mg/kg, about 5 mg/kg to about 5,000 mg/kg, about 10 mg/kg to about 3,000 mg/kg. In one embodiment, Coenzyme Q10 is administered in the range of about 10 mg/kg to about 1,400 mg/kg. In one embodiment, Coenzyme Q10 is administered in the range of about 10 mg/kg to about 650 mg/kg. In one embodiment, Coenzyme Q10 is administered in the range of about 10 mg/kg to about 200 mg/kg. In various embodiments, Coenzyme Q10 is administered at a dose of about 2mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 58 mg/kg, 58.6 mg/kg, 60 mg/kg, 65 mg/kg, 70 mg/kg, 75 mg/kg, 78 mg/kg, 80 mg/kg, 85 mg/kg, 90 mg/kg, 95 mg/kg, 100 mg/kg, 104 mg/kg, 110 mg/kg, 120 mg/kg, 130 mg/kg, 140 mg/kg, 150 mg/kg, 160 mg/kg, 170 mg/kg, 180 mg/kg, 190 mg/kg or 200 mg/kg. It should be understood that ranges having any one of these values as the upper or lower limits are also intended to be part of this invention, e.g., about 50 mg/kg to about 200 mg/kg, or about 650 mg/kg to about 1400 mg/kg. In one embodiment the administered dose is at least about 1 mg/kg, at least about 5 mg/kg, at least about 10 mg/kg, at least about 12.5 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 58 mg/kg, at least about 58.6 mg/kg, at least about 60 mg/kg, at least about 75 mg/kg, at least about 78 mg/kg, at least about 100 mg/kg, at least about 104 mg/kg, at least about 125 mg/kg, at least about 150 mg/kg, at least about 175 mg/kg, at least about 200 mg/kg, at least about 300 mg/kg, or at least about 400 mg/kg.

In certain embodiments, the CoQ10 is administered in at least one dose per day. In certain embodiments, the CoQ10 is administered in at least two doses per day. In certain embodiments, the CoQ10 is administered in at least three dose per day. In certain embodiments, the CoQ10 is administered in one dose per day. In certain embodiments, the CoQ10 is administered in two doses per day. In certain embodiments, the CoQ10 is administered in three doses per day. In certain embodiments, the CoQ10 is administered by continuous infusion.

For example, in some embodiments, the aforementioned methods comprise a regimen of intravenously administering CoQ10, e.g., at least about 50 mg/kg of CoQ10, once daily for 3 weeks, optionally with one week rest, and subsequently administering a fatty acid metabolism inhibitor. In other embodiments, the method comprises a regimen of intravenously administering CoQ10, e.g., at least about 75 mg/kg of CoQ10, once daily for 3 weeks, optionally with one week rest, and subsequently administering a fatty acid metabolism inhibitor.

Dosing ranges for inhaled formulations of CoQ10 can be similar to those provided for administration by injection. It is understood that nebulizers or other devices for delivery by inhalation are known in the art and can be used in conjunction with the methods of the invention.

Dosages of topical CoQ10 typically depend on the size of the area to be treated. For example, topically administered CoQ10 can be used for the treatment of skin cancer. CoQ10 is applied topically, typically once or twice per day, to the site of the cancerous lesion in an amount sufficient to cover the lesion, e.g., as applying acne medicine to a pimple. If the subject has many lesions for treatment, the CoQ10 is applied to many sites, increasing the total dose administered to the subject. If the subject has a single lesion, the CoQ10 is applied to the single site.

In general, the combination therapy including any of the CoQ10 formulations and the fatty acid metabolism inhibitors described herein may be used to prophylactically or therapeutically treat any neoplasm. In a particular embodiment, the combination therapy is used to treat solid tumors. In various embodiments of the invention, the combination therapy is used for treatment or prevention of cancer of the brain, central nervous system, head and neck, prostate, breast, testicular, pancreas, liver, colon, bladder, urethra, gall bladder, kidney, lung, non-small cell lung, melanoma, mesothelioma, uterus, cervix, ovary, sarcoma, bone, stomach, skin, and medulloblastoma. In a preferred embodiment, the combination therapy is used to treat breast cancer, for example, a triple -negative breast cancer (TNBC). In one embodiment, the combination therapy including CoQ10 described herein may be used to treat a chloroleukemia, e.g., a primary chloroleukemia or a secondary or metastatic chloroleukemia, e.g., that presents, migrates or metastasizes to a particular organ such as, e.g., the lung, the liver or the central nervous system.

However, treatment using combination therapies of the invention is not limited to the foregoing types of cancers. Examples of cancers amenable to treatment with the combination therapies include, but are not limited to, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, skin cancer, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer. In one embodiment, a CoQ10 IV formulation described herein may be used in combination with a fatty acid metabolism inhibitorto treat or prevent various types of skin cancer (e.g., Squamous cell Carcinoma or Basal Cell Carcinoma), pancreatic cancer, breast cancer, prostate cancer, liver cancer, or bone cancer. In one embodiment, the combination therapy including CoQ10 is used for treatment of a skin oncological disorder including, but not limited to, squamous cell carcinomas (including SCCIS (in situ) and more aggressive squamous cell carcinomas), basal cell carcinomas (including superficial, nodular and infiltrating basal cell carcinomas), melanomas, and actinic keratosis. In one embodiment, the oncological disorder or cancer which can be treated with the combination therapy including CoQ10 is not melanoma. In one embodiment, the oncological disorder is merkel cell carcinoma (MCC). In one embodiment, the oncological disorder or cancer which can be treated with the combination therapy including CoQ10 is not skin cancer.

In a particular embodiment, the oncological disorder is a tumor or cancer that is embedded in fatty tissue, for example, breast tissue or ovarian tissue. In certain embodiments, the oncological disorder is a tumor or cancer that utilizes or relies upon the fatty acids in the surrounding fatty tissue for growth. In certain embodiments, such an oncological disorder is selected from the group consisting of breast cancer, ovarian cancer, pancreatic cancer, and renal cell carcinoma. Such oncological disorders are expected to be particularly sensitive or responsive to the combination treatments of the invention.

In certain embodiments, the effect that combination therapy including CoQ10 may have on cancer cells may depend, in part, on the various states of metabolic and oxidative flux exhibited by the cancer cells. CoQ10 may be utilized to interrupt and/or interfere with the conversion of an oncogenic cell's dependency of glycolysis and increased lactate utility. As it relates to a cancer state, this interference with the glycolytic and oxidative flux of the tumor microenvironment may influence apoptosis and angiogenesis in a manner which reduces the development of a cancer cell. In some embodiments, the interaction of CoQ10 with glycolytic and oxidative flux factors may enhance the ability of CoQ10 to exert its restorative apoptotic effect in cancer while establishing viable drug targets for drug discovery and development.

In one embodiment, administration of CoQ10 and the fatty acid metabolism inhibitor as described or incorporated herein, reduces tumor size, weight or volume, increases time to progression, inhibits tumor growth and/or prolongs the survival time of a subject having an oncological disorder. In a preferred embodiment, CoQ10 is administered by injection, e.g., by intravenous administration, of an intravenous CoQ10 formulation as described or incorporated herein. In certain embodiments, administration of CoQ10 and the fatty acid metabolism inhibitor reduces tumor size, weight or volume, increases time to progression, inhibits tumor growth and/or prolongs the survival time of the subject by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is administered CoQ10 alone or the fatty acid metabolism inhibitor alone. In other embodiments, administration of CoQ10 and the fatty acid metabolism inhibitor stabilizes the oncological disorder in a subject with a progressive oncological disorder prior to treatment.

This invention also relates to a method of treating tumors in a human or other animal by intravenously administering to such human or animal an effective, non-toxic amount of CoQ10. One skilled in the art would be able, by routine experimentation, to determine what an effective, non-toxic amount of CoQ10 would be for the purpose of treating malignancies. For example, a therapeutically active amount of CoQ10 may vary according to factors such as the disease stage (e.g., stage I versus stage IV), age, sex, medical complications (e.g., immunosuppressed conditions or diseases) and weight of the subject, and the ability of the CoQ10 to elicit a desired response in the subject. The dosage regimen may be adjusted to provide the optimum therapeutic response. For example, several divided doses may be administered daily or administered by continuous infusion or the dose may be proportionally reduced as indicated by the exigencies of the therapeutic situation.

The invention also provides, in another aspect, methods for treating or preventing aggressive oncological disorders in humans. These methods include intravenously administering CoQ10 to the human at a therapeutically effective dose while co-administering a fatty acid metabolism inhibitor, so that treatment or prevention of the aggressive oncological disorder occurs. In one embodiment, these methods include intravenously administering CoQ10 to the subject at a selected lower dosage than a dosage regimen used or selected for less aggressive or non-aggressive oncological disorder, so that treatment or prevention of the aggressive oncological disorder occurs. In certain embodiments the aggressive oncological disorder includes pancreatic carcinoma, hepatocellular carcinoma, Ewing's sarcoma, metastatic breast cancer, metastatic melanoma, brain cancer (astrocytoma, glioblastoma), neuroendocrine cancer, colon cancer, liver cancer, lung cancer, osteosarcoma, androgen-independent prostate cancer, ovarian cancer, skin cancer, and non-Hodgkin's Lymphoma.

In another aspect, the invention provides methods for topical administration of CoQ10, especially in the treatment of skin cancer, in combination with administration of fatty acid metabolism inhibitors by any route of administration. Such methods include pre-treatment with CoQ10 prior to first administration of the fatty acid metabolism inhibitor.

In a related aspect, the invention provides a method for treating or preventing a non-aggressive oncological disorder in a human. These methods include intravenously co-administering CoQ10 and a fatty acid metabolism inhibitor to the subject at a therapeutically effective dose, so that treatment or prevention of the non-aggressive oncological disorder occurs. In one embodiment, these methods include administering CoQ10 to the subject at a selected higher dosage over a dosage regimen used or selected for aggressive oncological disorders so that treatment or prevention of the non-aggressive oncological disorder occurs. In certain embodiments, the non-aggressive oncological disorder includes non-metastatic breast cancer, androgen-dependent prostate cancer, small cell lung cancer and acute lymphocytic leukemia.

In some embodiments the oncological disorder is selected from the group consisting of leukemia, a lymphoma, a melanoma, a carcinoma, and a sarcoma.

International Patent Application Publication No. WO/2009/126764, filed Apr. 9, 2009, discloses the treatment of cancer with CoQ10 and International Patent Application Publication No. WO2011/11290, filed Mar. 11, 2011, discloses intravenous formulations of CoQ10. US Patent Application Publication No.: US2011/0027247 filed May, 11, 2010, discloses methods of treating oncological disorders using topically administered CoQ10. International Patent Application Nos. WO2009073843, filed Jun. 11, 2009, and WO2012174559, filed Jun. 18, 2012 disclose formulations of CoQ10 for administration by inhalation. These applications are each hereby incorporated by reference in their entirety. In certain embodiments of the invention, the methods further include a treatment regimen which includes any one of or a combination of surgery, radiation, hormone therapy, antibody therapy, therapy with growth factors, cytokines, and chemotherapy.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

Incorporation by Reference

Each reference, patent, and patent application referred to in the instant application is hereby incorporated by reference as if each reference were noted to be incorporated individually.

### EXAMPLES

The following example provides non-limiting exemplary methods and results from the treatment of cancer cells with the combination therapy of CoQ10 and fatty acid metabolism inhibitors.

### Example 1

### Effects of Coenzyme Q10 Alone or in Combination with Fatty Acid Metabolism Inhibitors on Cell Viability, Fatty Acid Metabolism Gene Expression, Glycerol Content and Fatty Acid Composition in Breast Cancer Cells

Cell viability was assessed in MDA-MB231 and SkBr-3 breast cancer cells exposed to Coenzyme Q10 alone or in combination with the fatty acid metabolism inhibitors C75, etomoxir, and trimetazidine, as well as a more pleiotropic fatty acid metabolism modulator, metformin. C75, etomoxir, and trimetazidine are selective inhibitors of specific enzymes in the fatty acid synthesis/metabolism pathway (fatty acid synthase (FASN), carnitine palmitoyltransferase I (CPT1), and long-chain 3-ketoacyl-CoA thiolase, respectively). Metformin is used clinically as an anti-diabetic agent to suppress glucose production by the liver; however, the mechanism of action remains incompletely understood. Metformin has multiple targets, all of which may contribute to its glucose lowering activity (e.g., mitochondrial complex I inhibitor, AMPK activator, regulation of PKA signaling, and others).

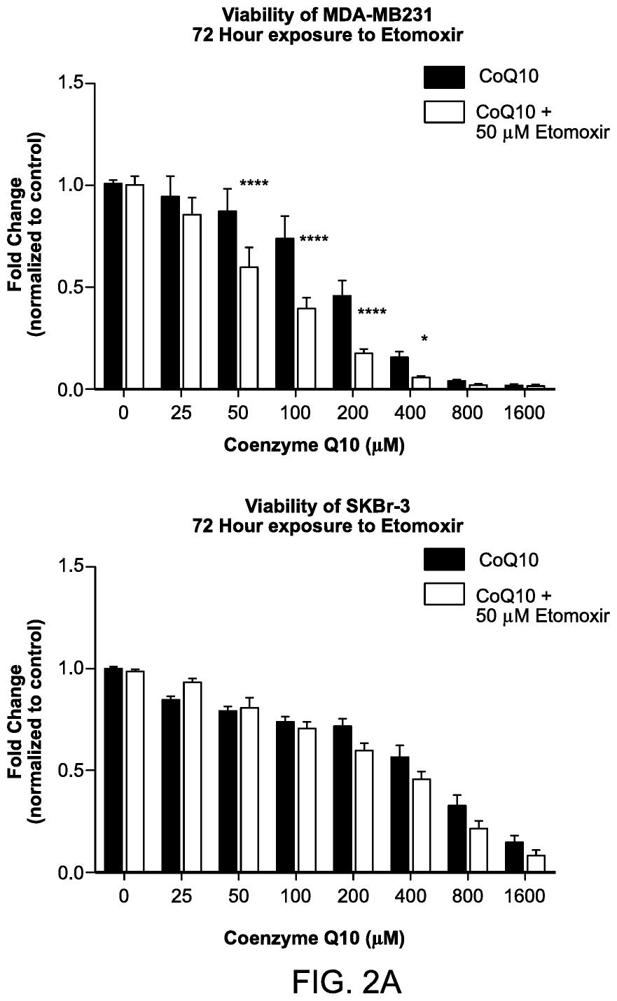

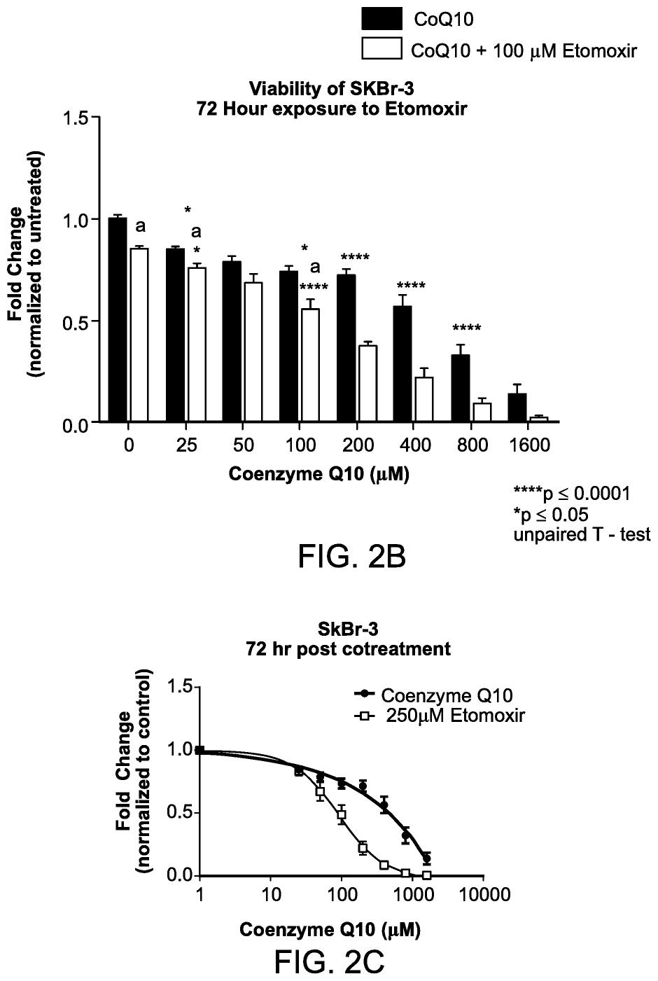

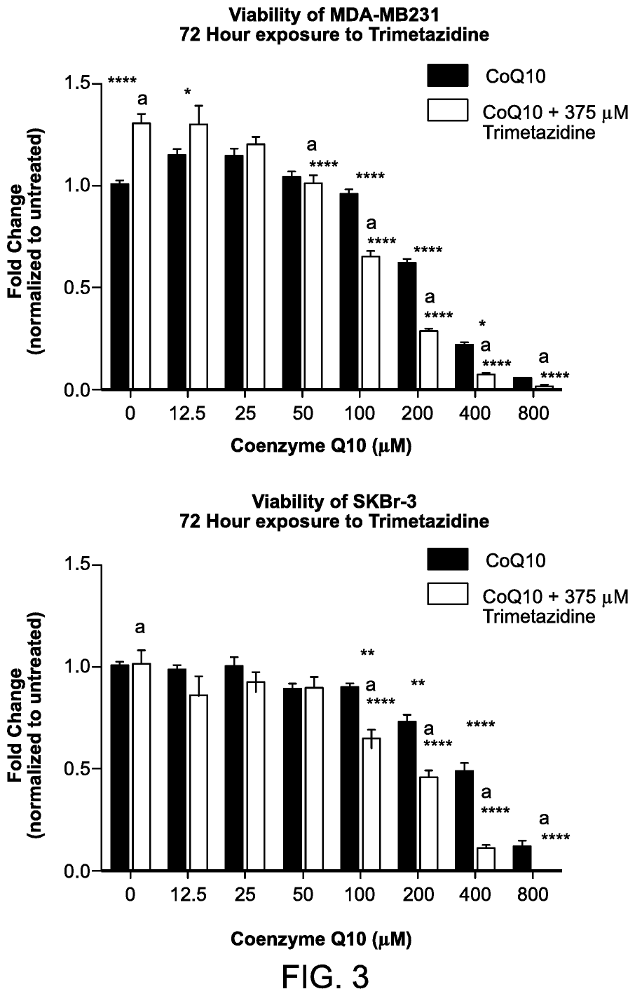

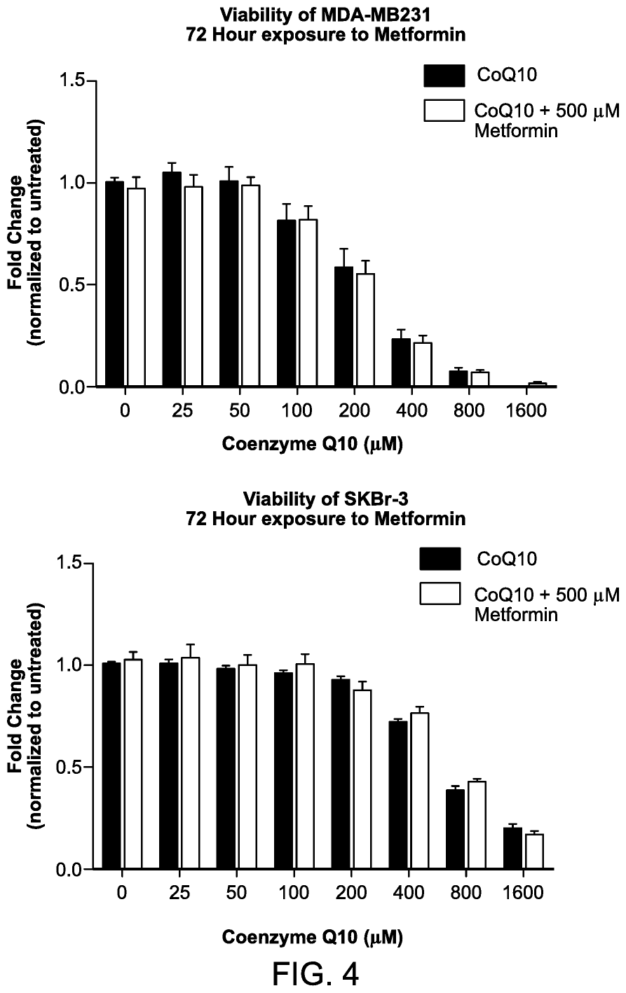

Coenzyme Q10 was applied to the cells in a 4% Coenzyme Q10 nanosuspension formulation (as described in WO 2011/112900, the entire contents of which are expressly incorporated herein by reference) comprising a phosphatidylcholine (e.g., dimyristoylphosphatidyl choline (DMPC)). Results of the experiments demonstrate that breast cancer cells were more sensitive to Coenzyme Q10 when treated in combination with C75 (FIG. 1), etomoxir (FIG. 2), and trimetazidine (FIG. 3) as indicated by a left-shift in the Coenzyme Q10 dose-response curve in both MDA-MB231 and SkBr-3 cells. These experiments were carried out at doses of fatty acid metabolism inhibitors which resulted in minimal or no cytotoxicity when the cells were treated with the fatty acid metabolism inhibitor alone (see bars corresponding to fatty acid metabolism inhibitor treatment and with 0 μM CoQ10). The effect of the combination treatments was generally observed to be synergistic. In contrast, combined treatment with metformin did not alter cytotoxic responses to Coenzyme Q10 (FIG. 4), indicating specificity of the synergistic effect for responses in fatty acid metabolism pathway modulation. The EC₅₀ for each treatment group is shown in the table below.

| MDA-MB231 | | MDA-MB231 | SKBr-3 |
|---|---|---|---|
| Treatment | Target | EC_{50(μm)} | EC_{50(μm)} |
| Coenzyme Q10 | | 165 | 599 |
| +Etomoxir | CPT-1 | 69 | 92 |
| +Trimetazidine | β-oxidation | 100 | 206 |
| +C75 | FASN | 113 | 324 |
| +Metformin | pleiotropic | 218 | 746 |

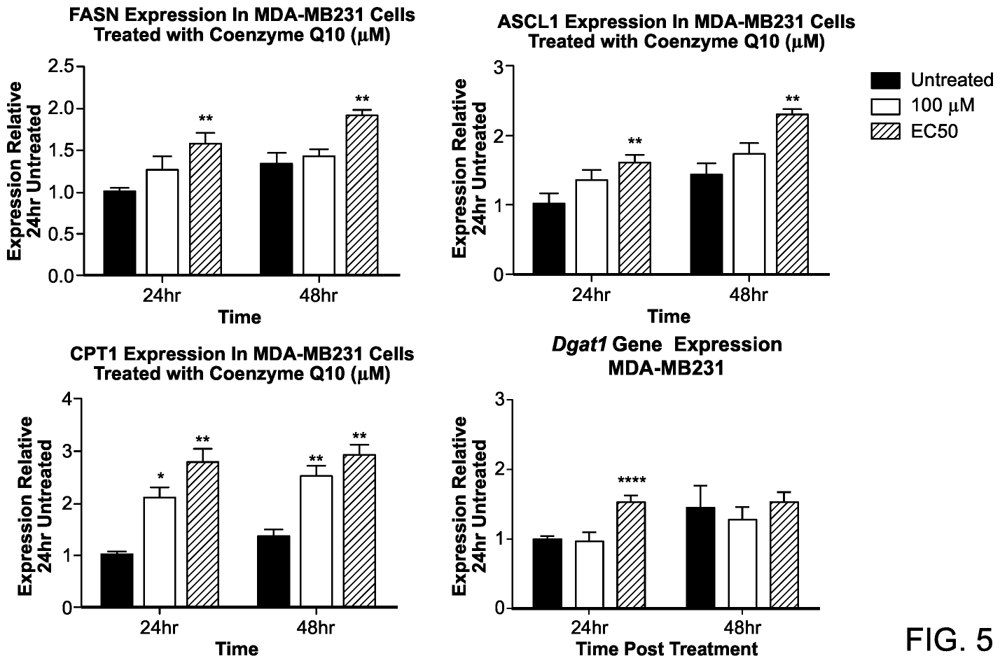

In addition, results from these studies showed that Coenzyme Q10 treatment alone was associated with a dose- and time-dependent increase in mRNA expression of the fatty acid metabolism gene products fatty acid synthase (FASN), carnitine palmitoyltransferase 1 (CPT-1), long-chain-fatty-acid—CoA ligase 1 (ACSL1), and diglyceride acyltransferase (DGAT1) in MDA-MB231 cells (FIG. 5). FASN catalyzes the synthesis of the fatty acid palmitate (C16:0, a long-chain saturated fatty acid) from acetyl-CoA and malonyl-CoA, in the presence of NADPH. CPT-1 is a mitochondrial enzyme responsible for the formation of acyl carnitines by catalyzing the transfer of the acyl group of a long-chain fatty acyl-CoA from coenzyme A to l-carnitine. CPT-1 mediated formation of acyl carnitine allows for subsequent movement of the acyl carnitine from the cytosol into the intermembrane space of mitochondria. ACSL1 activates the degradation of complex fatty acids. Specifically, this enzyme catalyzes the formation of fatty acyl-CoA by a two-step process proceeding through an adenylated intermediate. DGAT1 catalyzes the formation of triglycerides from diacylglycerol and Acyl-CoA. The reaction catalyzed by DGAT is considered the terminal and only committed step in triglyceride synthesis and to be essential for the formation of adipose tissue. Accordingly, these results indicate that Coenzyme Q10 treatment of cancer cells results in changes in several aspects of fatty acid metabolism such as fatty acid synthesis, transport, storage and degradation.

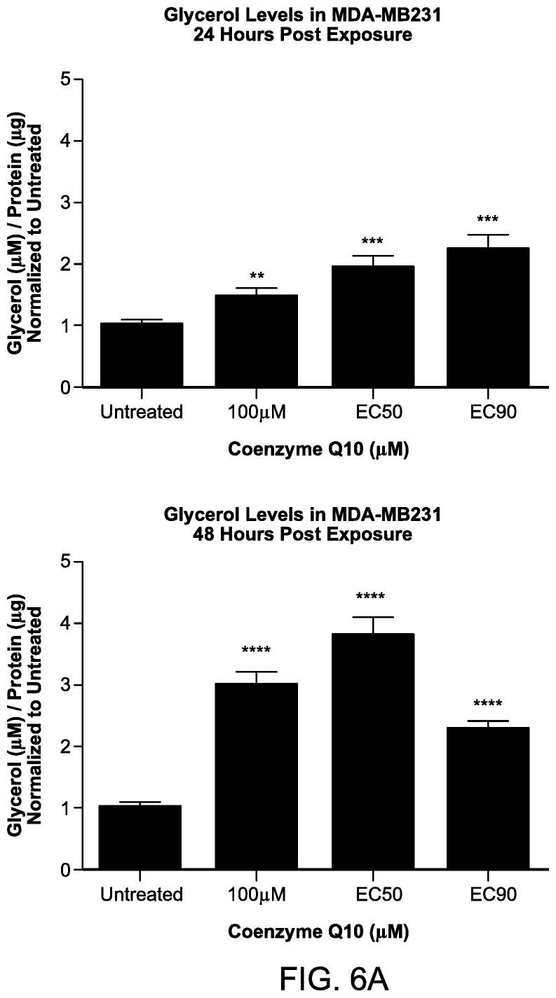

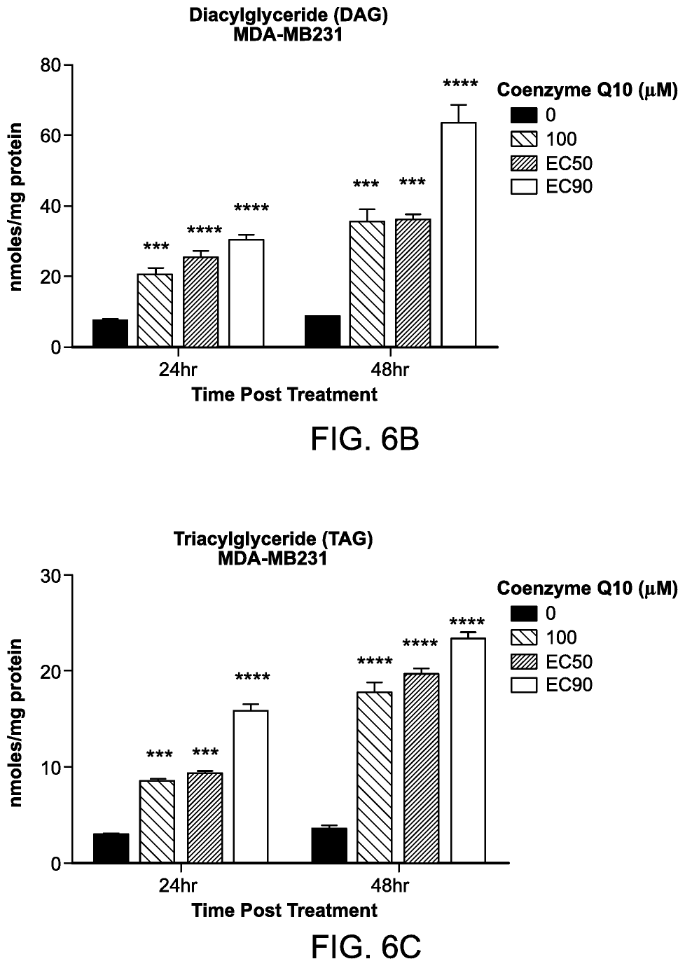

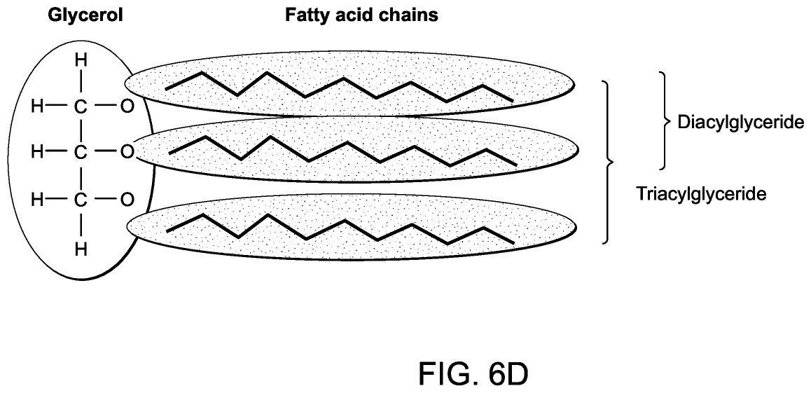

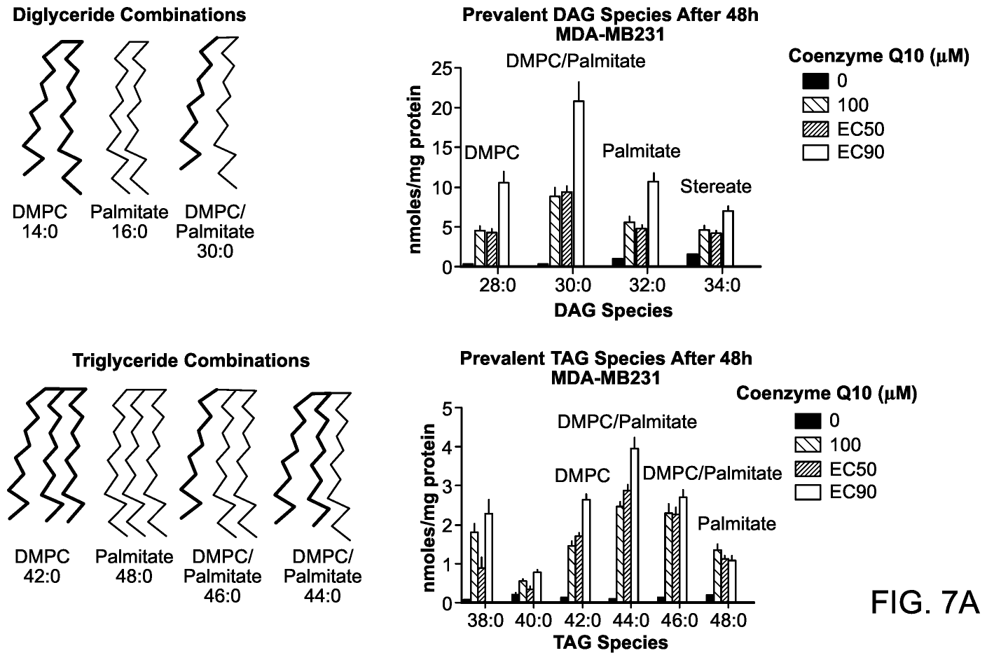

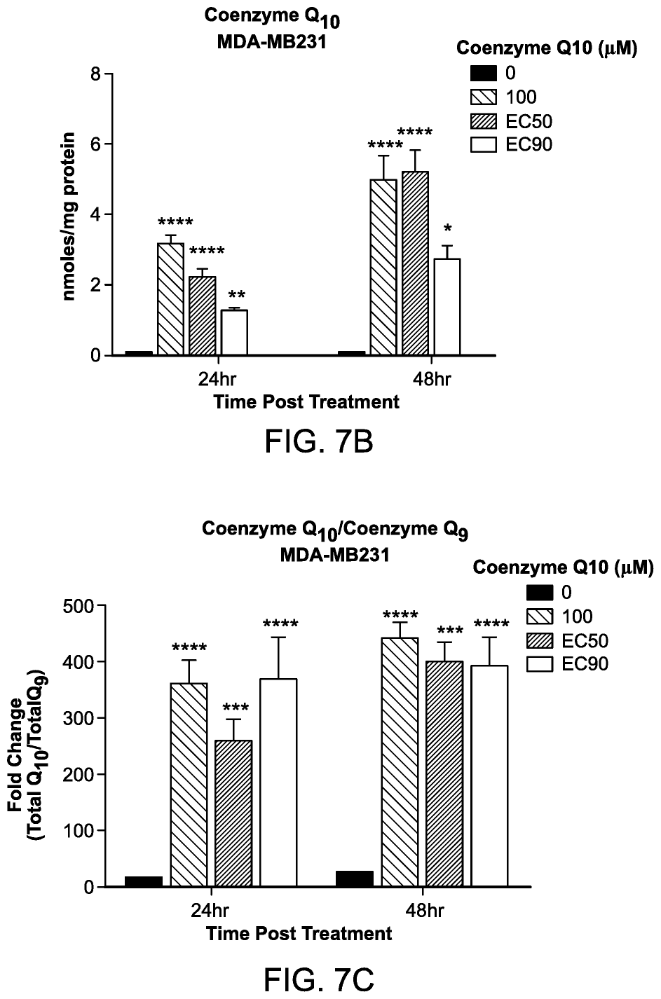

In further experiments, Coenzyme Q10 treatment was observed to result in accumulation of diacylglyceride (DAG), triacylglyceride (TAG) and the triglyceride backbone, glycerol, in MDA-MB231 cells (FIG. 6). To further evaluate the effect of Coenzyme Q10 on cancer cell lipid composition, structural lipidomic analysis was used to assess the metabolic fate of the lipid components of the Coenzyme Q10 formulation itself, such as DMPC. DMPC is a 14:0 lipid which is not found endogenously in human cells. Accordingly, it was possible to distinguish between DMPC-containing species based on the length of the fatty acid chains, e.g. 28:0 DAG and 42:0 TAG are not found endogenously. Structural lipodomic analysis demonstrated that these lipid components were readily incorporated into prevalent diacyl-glyerol (DAG) and triacyl-glycerol (TAG) species along with de novo fatty acid species such as palmitate (FIG. 7A) in MDA-MB231 cells. These results demonstrated that lipid components of the Coenzyme Q10 formulation employed in the experiments were readily accumulated in cancer cells and incorporated into DAG and TAG species, thus indicating that cancer cells actively respond to the addition of exogenous lipids to increase their storage or degradation. Accordingly, cancer cells treated with lipid-containing formulations may be especially sensitive to fatty acid metabolism inhibitors, due to the increased fatty acid metabolism activity resulting from storage and degradation of the lipid formulation components.

Coenzyme Q10 levels in breast cancer cells treated with Coenzyme Q10 were also evaluated. FIG. 7B shows that treatment of MDA-MB231 cells with Coenzyme Q10 significantly increased the amount of Coenzyme Q10 in these cells at 24 and 48 hours after treatment. FIG. 7C shows that Coenzyme treatment also significantly increased the ratio of Coenzyme Q10 to endogenous Coenzyme Q9 in MDA-MB231 cells 24 and 48 hours after treatment.

Together these results demonstrate that, in general, combination treatment with Coenzyme Q10 and a fatty acid metabolism inhibitor is more effective than treatment with Coenzyme Q10 treatment or a fatty acid metabolism alone. The results also indicate that the fatty acid metabolism inhibitors and Coenzyme Q10 act synergistically to cause cancer cell death. Finally, these results demonstrate that Coenzyme Q10 treatment itself alters fatty acid gene expression and endogenous and exogenous lipid homeostasis in breast cancer cells.

### Example 2

### Effects of Coenzyme Q10 Alone or in Combination with Fatty Acid Metabolism Inhibitors on Cell Viability in Pancreatic Cancer Cells

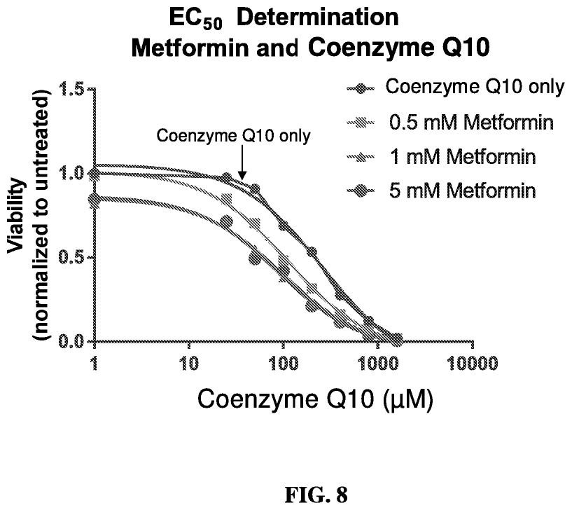

Cell viability was next assessed in MIA PaCa-2 pancreatic cancer cells exposed to Coenzyme Q10 alone or in combination with the fatty acid metabolism inhibitors C75, etomoxir, trimetazidine, metformin, and the Stearoyl-CoA desaturase-1 (SCD-1) inhibitor A939572. The experiments were generally carried out similarly as the experiments described in Example 1. SCD-1 is an endoplasmic reticulum enzyme that catalyzes the formation of the monounsaturated fatty acids oleic acid and palmitoleic acid from stearoyl-CoA and palmitoyl-CoA. Oleic acid and palmitoleic acid are major components of membrane phospholipids, cholesterol esters and alkyl-diacylglycerol. Thus SCD-1 is a key enzyme in fatty acid metabolism. Inhibition of SCD-1 results in depletion of mono-unsaturated fatty acids and reduction of lipid storage. Results from these studies showed that pancreatic cancer cells were more sensitive to Coenzyme Q10 when treated in combination with metformin, as shown by reduced viability of cells treated with the combination (See FIG. 8). Pancreatic cancer cells treated with Coenzyme Q10 and 0.5 mM metformin exhibited lower viability than cells treated with Coenzyme Q10 alone, and cells treated with Coenzyme Q10 and 1 mM or 5 mM metformin had lower viability than cells treated with Coenzyme Q10 and 0.5 mM metformin. The EC₅₀ for Coenzyme Q10 alone and in combination with various concentrations of metformin in the pancreatic cancer cells is shown in the table below.

| Treatment | EC₅₀ | 95% C.I. |
|---|---|---|
| Coenzyme Q10 | 260.8 | 211.5 to 323.9 |
| +0.5 mM Metformin | 112.4 | 92.99 to 136.5 |
| +1 mM Metformin | 97.75 | 75.23 to 128.4 |
| +5 mM Metformin | 90.67 | 69.56 to 119.4 |

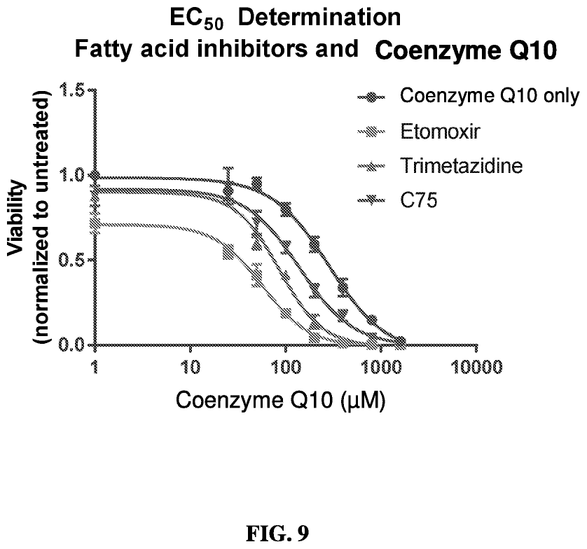

As shown in FIG. 9, pancreatic cancer cells were also more sensitive to Coenzyme Q10 when treated in combination with Etomoxir, C75 or Trimetazidine, as shown by reduced viability of cells treated with the combinations. The EC₅₀ for Coenzyme Q10 alone and in combination with Etomoxir (100 μM), Trimetazidine (250 μM) or C75 (10μ) in pancreatic cancer cells is shown in the table below.

| Treatment | EC₅₀ | 95% C.I. |
|---|---|---|
| BPM 31510 | 284.4 | 223.7 to 411.2 |
| +100 μM Etomoxir | 56.77 | 45.33 to 71.08 |
| +10 μM C75 | 139.3 | 101.8 to 202.3 |
| +250 μM Trimetazidine | 85.87 | 75.72 to 97.47 |

These results demonstrate that blocking fatty acid synthesis (C75), transport of fatty acids into the mitochondria (Etomoxir), or beta-oxidation (Trimetazidine) sensitizes MIA PaCa-2 pancreatic cancer cells to Coenzyme Q10 treatment.

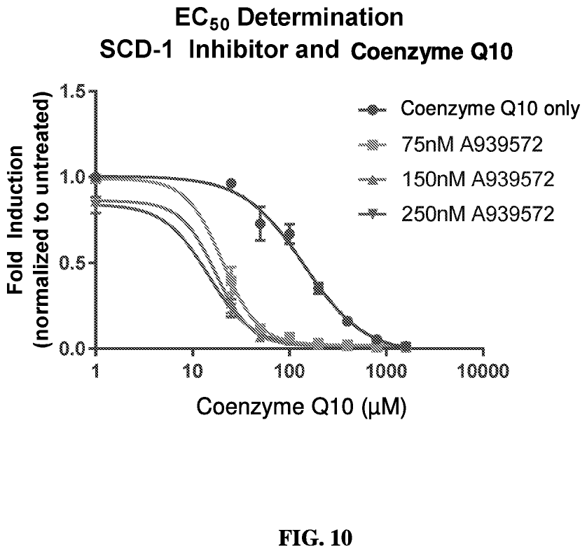

Finally, as shown in FIG. 10, pancreatic cancer cells were more sensitive to Coenzyme Q10 when treated in combination with the SCD-1 inhibitor A939572. The EC₅₀ for Coenzyme Q10 alone and in combination with various concentrations of the SCD-1 inhibitor A939572 in pancreatic cancer cells is shown in the table below.

| Treatment | EC₅₀ | 95% C.I. |
|---|---|---|
| Coenzyme Q10 | 141.5 | 108.9 to 190.3 |
| +75 nM A939572 | 20.44 | 16.2 to 23.27 |
| +150 nM A939572 | 17.38 | 11.8 to 21.52 |
| +250 nM A939572 | 14.56 | 5.233 to 20.61 |

Because inhibition of SCD-1 results in depletion of mono-unsaturated fatty acids and reduction of lipid storage, these results indicate that reducing the ability of a cancer cell to store lipids increases the sensitivity of the cancer cell to Coenzyme Q10.

1. A method of treating an oncological disorder in a subject comprising administering Coenzyme Q10 (CoQ10) and at least one fatty acid metabolism inhibitor to the subject, thereby treating the oncological disorder in the subject.

2. The method of claim 1, wherein a response of the oncological disorder to treatment is improved relative to treatment with the at least one fatty acid metabolism inhibitor alone.

3. The method of claim 1 or 2, wherein a response of the oncological disorder to treatment is improved relative to treatment with CoQ10 alone.

4. The method of claim 2 or 3, wherein the response is improved by at least 5%, at least 10%, at least 15%, at least 20%, at least 30%, at least 40% or at least 50% relative to treatment with the at least one fatty acid metabolism inhibitor alone.

5. The method of any one of claims 2 to 4, wherein the response is improved by at least 5%, at least 10%, at least 15%, at least 20%, at least 30%, at least 40% or at least 50% relative to treatment with Coenzyme Q10 alone.

6. The method of any one of claims 2 to 5, wherein the response comprises any one or more of reduction in tumor burden, reduction in tumor size, inhibition of tumor growth, achieving stable oncological disorder in a subject with a progressive oncological disorder prior to treatment, increased time to progression of the oncological disorder, and increased time of survival.

7. A method of treating an oncological disorder in a subject comprising:
(a) administering Coenzyme Q10 (CoQ10) to the subject; and
(b) administering at least one fatty acid metabolism inhibitor to the subject at a dosage that is lower than standard dosages of the fatty acid metabolism inhibitor used to treat the oncological disorder, thereby treating the oncological disorder.

8. A method of treating an oncological disorder in a subject comprising:
(a) administering at least one fatty acid metabolism inhibitor to the subject; and
(b) administering Coenzyme Q10 (CoQ10) to the subject at a dosage that is lower than standard dosages of the CoQ10 used to treat the oncological disorder, thereby treating the oncological disorder.

9. The method of any one of claims 1, 7 and 8, wherein the CoQ10 is administered topically.

10. The method of any one of claims 1, 7 and 8, wherein the CoQ10 is administered by inhalation.

11. The method of any one of claims 1, 7 and 8, wherein the CoQ10 is administered by injection or infusion.

12. The method of any one of claims 1, 7 and 8, wherein the CoQ10 is administered by intravenous administration.

13. The method of claim 12, wherein the CoQ10 is administered by continuous intravenous infusion.

14. The method of any one of claims 1 to 13, wherein the CoQ10 is formulated in a lipid composition.

15. The method of claim 14, wherein the lipid composition comprises:
an aqueous solution, CoQ10, and at least one of a dispersion stabilizing agent and an opsonization reducer; wherein the CoQ10 is dispersed into a colloidal nano-dispersion of particles having a mean particle size of less than 200 nm.

16. The method of claim 15, wherein the dispersion stabilizing agent is selected from the group consisting of pegylated castor oil, Cremophor EL, Cremophor RH 40, Pegylated vitamin E, Vitamin E TPGS, and Dimyristoylphosphatidyl choline (DMPC).

17. The method of claim 16, wherein the dispersion stabilizing agent is DMPC.

18. The method of any one of claims 1 to 17, wherein the at least one fatty acid metabolism inhibitor comprises a nucleic acid, a polypeptide, or a small molecule.

19. The method of claim 18, wherein the fatty acid metabolism inhibitor is a nucleic acid inhibitor.

20. The method of claim 19, wherein the nucleic acid inhibitor is an antisense nucleic acid molecule or a double stranded nucleic acid molecule.

21. The method of claim 20, wherein the double stranded nucleic acid molecule is a double stranded RNA selected from the group consisting of an siRNA, a shRNA, and a dicer substrate siRNA (DsiRNA).

22. The method of claim 18, wherein the fatty acid metabolism inhibitor is a polypeptide.

23. The method of claim 22, wherein the polypeptide is an antibody or an antigen-binding fragment thereof.

24. The method of claim 18, wherein the at least one fatty acid metabolism inhibitor comprises a small molecule.

25. The method of claim 24, wherein the small molecule is a carboxylic acid.

26. The method of any one of claims 1 to 25, wherein the at least one fatty acid metabolism inhibitor is an inhibitor of a process in fatty acid metabolism selected from the group consisting of fatty acid synthesis, fatty acid transport and storage, fatty acid degradation, and a modulator of fatty acid structure.

27. The method of any one of claims 1 to 26, wherein the at least one fatty acid metabolism inhibitor is an inhibitor of a molecule selected from the group consisting of fatty acid synthase (FASN), carnitine palmitoyltransferase 1 (CPT-1), long-chain 3-ketoacyl-CoA thiolase, and stearoyl-CoA desaturase-1 (SCD-1).

28. The method of claim 27, wherein the at least one fatty acid metabolism inhibitor is an inhibitor of FASN.

29. The method of claim 28, wherein the inhibitor of FASN is C75.

30. The method of claim 27, wherein the at least one fatty acid metabolism inhibitor is an inhibitor of CPT-1.

31. The method of claim 30, wherein the inhibitor of CPT-1 is etomoxir.

32. The method of claim 22, wherein the at least one fatty acid metabolism inhibitor is an inhibitor of long-chain 3-ketoacyl-CoA thiolase.

33. The method of claim 32, wherein the inhibitor of long-chain 3-ketoacyl-CoA thiolase is trimetazidine.

34. The method of claim 27, wherein the at least one fatty acid metabolism inhibitor is an inhibitor of SCD-1.

35. The method of claim 34, wherein the inhibitor of SCD-1 is A939572.

36. The method of claim 1, wherein the at least one fatty acid metabolism inhibitor is selected from the group consisting of etomoxir, C75, trimetazidine, A939572, cerulenin, 5-(tetradecyloxy)-2-furoic acid, oxfenicine, methyl palmoxirate, metoprolol, amiodarone, perhexiline, aminocarnitine, hydrazonopropionic acid, 4-bromocrotonic acid, ranolazine, hypoglycin, dichloroacetate, methylene cyclopropyl acetic acid, beta-hydroxy butyrate, and a non-hydrolyzable analog of carnitine, and the compounds listed in Tables 2A, 2B, 2C, 2D, 3 or 4.

37. The method of claim 1, wherein the at least one fatty acid metabolism inhibitor is an oxirane carboxylic acid compound having the following formula: 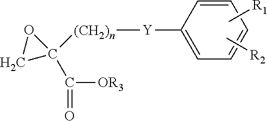
wherein: R1 represents a hydrogen atom, a halogen atom, a 1-4C alkyl group, a 1-4C alkoxy group, a nitro group or a trifluoromethyl group; R2 has one of the meanings of R1; R3 represents a hydrogen atom or a 1-4C alkyl group; Y represents the grouping —O—(CH2)m-;
m is 0 or a whole number from 1 to 4; and n is a whole number from 2 to 8 wherein the sum of m and n is a whole number from 2 to 8.

38. The method of claim 37, wherein R1 is a halogen atom, R2 is a hydrogen atom, m is 0, and n is 6, and R3 is an ethyl group.

39. The method of claim 1, wherein the fatty acid metabolism inhibitor is selected from the group consisting of 2-(6-(4-chlorophenoxy)-hexyl)-oxirane-2-carboxylic acid ethyl ester (etomoxir), 2-(4-(3-chlorophenoxy)-butyl)-oxirane-2-carboxylic acid ethyl ester, 2-(4-(3-trifluoromethylphenoxy)-butyl)-oxirane-2-carboxylic acid ethyl ester, 2-(5(4-chlorophenoxy)-pentyl)-oxirane-2-carboxylic acid ethyl ester, 2-(6-(3,4-dichlorophenoxy)-hexyl)-oxirane-2-carboxylic acid ethyl ester, 2-(6-(4-fluorophenoxy)-hexyl)-oxirane-2-carboxylic acid ethyl ester, and 2-(6-phenoxyhexyl)-oxirane-2-carboxylic acid ethyl ester, the corresponding oxirane carboxylic acids, and their pharmacologically acceptable salts.

40. The method of claim 1, wherein the at least one fatty acid metabolism inhibitor has the structure: 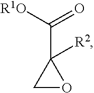
wherein one or both of R1 and R2 are independently an alkyl.

41. The method of claim 40, wherein the alkyl is a straight-chain alkyl.

42. The method of claim 40, wherein R2 has the structure: 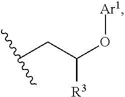
where R3 comprises an organic moiety and Ar1 comprises an aromatic moiety.

43. The method of claim 42, wherein Art is a benzene ring or a derivative thereof having the structure: 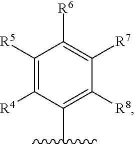
wherein each of R4, R5, R6, R7, and R8 is hydrogen, a halogen, an alkyl, or an alkoxy.

44. The method of claim 1, wherein the at least one fatty acid metabolism inhibitor has the structure: 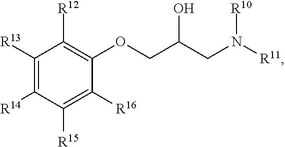
wherein each of R10, R11, R12, R13, R14 R15 and R16 independently comprises hydrogen, a halogen, or an organic moiety.

45. The method of claim 1, wherein the fatty acid metabolism inhibitor has the structure: 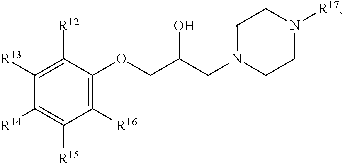
wherein R17 comprises an alkyl, an alkoxy, an aromatic moiety or an amide.

46. The method of any one of claims 1 to 45, further comprising administering at least one additional chemotherapeutic agent to the subject.

47. The method of any one of claims 1 to 46, wherein the oncological disorder is selected from the group consisting of a carcinoma, sarcoma, lymphoma, melanoma, and leukemia.

48. The method of any one of claims 1 to 46, wherein the oncological disorder is selected from the group consisting of pancreatic cancer, breast cancer, ovarian cancer, renal cell carcinoma, liver cancer, skin cancer, lung cancer, colon cancer, prostate cancer, thyroid cancer, bladder cancer, rectal cancer, endometrial cancer, kidney cancer, bone cancer, brain cancer, cervical cancer, stomach cancer, mouth and oral cancers, neuroblastoma, testicular cancer, uterine cancer, and vulvar cancer.

49. The method of claim 48, wherein the oncological disorder is selected from the group consisting of breast cancer, ovarian cancer, pancreatic cancer and renal cell carcinoma.

50. The method of any one of claims 1 to 49, wherein the subject is a human.
